(19) United States
(12) Patent Application Publication
(10) Pub. No.: US 2026/0253735 A1
(43) Pub. Date: Aug. 27, 2026

(54) HEALTH PREDICTION SYSTEM COMPRISING PERSONALIZED HEALTH PREDICTION LEARNING MODEL, AND CONTROL METHOD FOR SAME

(71) Applicant: LG ELECTRONICS INC., Seoul (KR)

(72) Inventors: Jingu CHOI, Seoul (KR); Sangwook PARK, Seoul (KR); Eunmin LIM, Seoul (KR); Taeyoung SONG, Seoul (KR); Byunghun CHOI, Seoul (KR)

(73) Assignee: LG ELECTRONICS INC., Seoul (KR)

(21) Appl. No.: 18/726,721

(22) PCT Filed: Dec. 29, 2022
(86) PCT No.: PCT/KR2022/021636

§ 371 Date: Jul. 3, 2024

(30) Foreign Application Priority Data
Jan. 5, 2022 (KR) 10-2022-0001760
Mar. 11, 2022 (KR) 10-2022-0030904
Aug. 19, 2022 (KR) 10-2022-0104093

(51) Int. Cl. G16H 50/30; G06F 21/62; G16H 20/60; G16H 40/67; G16H 50/20; G16H 50/50; H04L 9/00

(52) U.S. Cl. CPC G16H 50/30; G06F 21/6245; G16H 20/60; G16H 40/67; G16H 50/20; G16H 50/50; H04L 9/50

(57) ABSTRACT

A server of a health prediction system in one example includes a communication unit for performing wireless communication with a collection terminal for collecting user biometric information measured; an artificial intelligence unit which, when characteristic information about at least one measurement apparatus, generates a prediction model for a specific disease, and trains the prediction model on the basis of the collected user biometric information; and a control unit which, when the prediction model is trained, controls the artificial intelligence unit so that a prediction level related to the specific disease is calculated from the trained prediction model, determines a prediction result for the specific disease on the basis of the calculated prediction level and a preset threshold value, and transmits the determined prediction result to the collection terminal.

### TECHNICAL FIELD

The present disclosure relates to a health prediction system for diagnosing a user's health status and predicting a disease or the like, and more particularly, to a health prediction system provided with a personalized health prediction learning model specialized for the user through learning.

### BACKGROUND ART

At present, a variety of medical devices have been emerged due to the development of science and technology. In particular, with the advent of wearable devices that can be worn by a user, wearable devices with medical functions that diagnose the user's health status have been emerged in large numbers by taking into account that the wearable device is worn by the user.

As the medical devices have emerged in large numbers, a healthcare platform that provides the user' health information collected from the medical devices has emerged. Such a healthcare platform collects the collected medical information and provides the collected information to the user, thereby allowing the user to check information on his or her own health status.

However, as medical devices are diversified due to having emerged in large numbers, devices that are manufactured by different manufacturers or have different attachment or measurement types have emerged even though the devices measure the same health status. Furthermore, due to differences in manufacturing, attachment type, or measurement type, problems occur in which different diagnosis results are output depending on the characteristics of measurement devices even though the same health status of the user is checked. Accordingly, despite the same health status of the same user, the diagnosis results of the healthcare platform vary depending on which manufacturer's device is used, which type is used, and how the check is performed, which reduces the reliability of the diagnosis results.

In addition, in the case of medical diagnosis information, the influence of an individual user's biological characteristics may be very significant. Therefore, a customized diagnostic system personalized to a user is required, but there is a problem in that the current health care platform only provides a diagnosis result determined based on preset thresholds of collected data and does not provide such a customized diagnosis service.

Additionally, medical information, which is information on the physical characteristics of an individual user, may be very sensitive personal information. Therefore, it is very important to maintain the security of such medical information to protect personal privacy.

However, such medical information must be disclosed to a designated third party when necessary. For example, in the case of a family member or the like, medical information may need to be shared, and in the case of a third party such as a doctor or health trainer who needs to check the user's health status, it may be necessary to share the medical information. That is, it is necessary for an authorized third party to share medical information.

Therefore, there is a need for a medical information record management method that can strictly manage a user's medical information record stored in the healthcare platform to prevent it from being leaked and ensure that it is provided only to the authorized third party.

### DISCLOSURE OF INVENTION

### Technical Problem

The present disclosure aims to solve the above-described problems and other problems, and an aspect of the present disclosure is to provide a health prediction system that can diagnose a user's health status and a likelihood of predicting a disease or the like from the user's biometric information through a user-customized health prediction learning model that reflects the characteristics of a measurement device that is used by the user to measure biometric information for medical diagnosis and a control method for the same.

Furthermore, another aspect of the present disclosure is to provide a health prediction system that can dynamically determine whether a disease has developed according to a user's health status depending on the user's context and a control method for the same.

In addition, still another aspect of the present disclosure is to provide a health prediction system that allows a diagnosis result for biometric information measured from a user to be provided in real time simultaneously with the measurement of the biometric information and a control method for the same.

Moreover, yet still another aspect of the present disclosure is to provide a health prediction system that can prevent medical information measured and stored from a user from being provided to a third party, and allow an authorized third party to view the medical information so as to allow the authorized third party to check the user's medical information and a control method for the same.

### Solution to Problem

In order to achieve the foregoing or other objectives, according to an aspect of the present disclosure, there is provided a server of a health prediction system, the server including a communication unit that performs wireless communication with a collection terminal that collects a user's biometric information measured by at least one measurement device, an artificial intelligence unit that generates, when the characteristic information of the at least one measurement device is received through the communication unit, a prediction model for a specific disease by reflecting the received characteristic information of the measurement device, and performs training on the prediction model based on the collected user's biometric information, and a controller that controls, when training on the prediction model is carried out, the artificial intelligence unit to calculate a prediction rate related to the specific disease in response to biometric information collected in the collection terminal from the trained prediction model, discriminates a prediction result for the specific disease based on the calculated prediction rate and a preset threshold, and transmits the prediction result to the collection terminal such that the discriminated prediction result is displayed through the collection terminal.

In one embodiment, the collection terminal may acquire information related to a user's context from at least one peripheral device around the user, analyze the user's context based on the acquired information, and transmit context information on the analyzed user's context to the server, wherein the server changes a threshold based on the context information, and discriminates a prediction result for the specific disease based on the changed threshold and the prediction rate.

In one embodiment, the at least one peripheral device may be information on at least one mobile terminal located around the user or an access point (AP) provided in a region where the user is located.

In one embodiment, the prediction model may be an artificial neural network including at least one exclusive layer, which is a hidden layer including weights applied to input biometric information classified into a specific group, and at least one common layer, which is a hidden layer including weights applied to all input biometric information, wherein the biometric information is input to either one of the exclusive layer or the common layer depending on whether it is classified into the specific group, and a prediction rate output as a result of the prediction model is output from any one of the at least one common layer.

In one embodiment, the controller may input the biometric information as the biometric information of the specific group into the prediction model according to the characteristic information of a measurement device that measures the biometric information.

In one embodiment, the characteristic information of the measurement device may include at lase one of a manufacturer of the measurement device, a kind of biometric information measured by the measurement device, a type of the measurement device, a mounting method in which the measurement device is mounted by a user, and a measurement method in which the user's biometric information is measured by the measurement device.

In one embodiment, the characteristic information of the measurement device may include quantified clinical accuracy that quantifies the clinical accuracy of the measurement device.

In one embodiment, the controller may update the prediction model when a preset update condition is satisfied, wherein the preset update condition is satisfied when a preset update cycle is expired or when a measurement device that measures the user's biometric information is added, replaced, or any one measurement device is removed.

In one embodiment, the controller may update the prediction model to change a weight assigned to at least one node among hidden layers constituting the prediction model, or to remove at least one of the hidden layers or further include a new hidden layer including nodes to which a new weight is assigned, and determine the changed weight based on the device characteristic information of the added, replaced or removed measurement device, or determine the hidden layer to be removed or the new hidden layer.

In one embodiment, the controller may generate at least one new prediction model according to the device characteristic information of the added, replaced, or removed measurement device, and calculate a statistical value of prediction rates calculated from the existing prediction model and the at least one new prediction model, respectively, with respect to the collected user's biometric information as the prediction rate.

In one embodiment, the controller may update the trained prediction model by combining at least one prediction model derived from a training process for the prediction model based on the collected user's biometric information with the trained prediction model.

In one embodiment, the controller may compare the weights of hidden layers corresponding to one another from the trained prediction model and the at least one derived model, respectively, and add nodes with different weights to combine the trained prediction model with the at least one derived model.

In one embodiment, when the measurement device is a measurement device that measures a user's blood glucose level, the prediction model may be a model that has trained a variation in the user's blood glucose level that varies depending on each nutrient, wherein when a user inputs a virtual diet through a collection terminal, the controller analyzes the nutrients of each food included in the input diet, calculates a variation in the user's total blood glucose level corresponding to the virtual diet based on a variation in the user's blood glucose level according to the analyzed nutrients, respectively, and an intake amount of each food that is input with the virtual diet, and transmits the calculated variation to the collection terminal in response to an input of the virtual diet.

In one embodiment, when the training of the prediction model is completed, the controller may determine a range of the minimum and maximum values of the user's biometric information that can be detected through the measurement device from the user's basic biometric characteristics, control the artificial intelligence unit to calculate the prediction rates of the prediction model corresponding to each biometric information included in the determined range of the minimum and maximum values of the biometric information, and transmit some of the calculated prediction rates to the collection terminal as a caching table.

In one embodiment, when the caching table is received from the server, the collection terminal may detect a prediction rate corresponding to a bio-signal collected from the measurement device from the caching table, discriminate a prediction result for the specific disease based on the prediction rate detected from the detected caching table and a threshold according to the context information, and output the discriminated prediction result.

In one embodiment, the server may further include a memory in which the user's biometric information collected through the communication unit is classified and stored according to a time at which the biometric information is measured and a kind of the biometric information, wherein the controller provides address information related to a storage area where the classified biometric information is stored in the memory in response to a user's request.

In one embodiment, the controller may register a token representing the authority of a third party to request the user's biometric information through a blockchain network, provide the address information to the third party at the user's request based on the registered token, and limit a scope of the user's biometric information that can be viewed by the third party and a validity period during which the user's biometric information can be viewed based on the token registered in the blockchain network.

In order to achieve the foregoing or other objectives, according to an aspect of the present disclosure, there is provided a method of controlling a health prediction system that provides a prediction result related to a specific disease based on a user's biometric information, the method including connecting, by a collection terminal of the health prediction system, to at least one measurement device through communication to measure the user's biometric information, and collecting the device characteristic information of the at least one measurement device, receiving, by a cloud server, the characteristic information of the measurement device collected from the collection terminal, and generating a prediction model for a specific disease on which the characteristic information of the measurement device is reflected, receiving, by the collection terminal, the user's biometric information collected by the at least one measurement device, receiving, by the cloud server, biometric information collected by the collection terminal, and training the prediction model based on the received biometric information, calculating, by the cloud server, when the training of the prediction model is completed, a prediction rate corresponding to the biometric information received from the collection terminal from the trained prediction model, and discriminating, by the cloud server or the collection terminal, a prediction result for the specific disease based on a preset threshold and the prediction rate, and outputting the discriminated prediction result.

In one embodiment, the characteristic information of the measurement device may include at least one of a manufacturer of the measurement device, a kind of biometric information measured by the measurement device, a type of the measurement device, a mounting method in which the measurement device is mounted by a user, and a measurement method in which the user's biometric information is measured by the measurement device.

In one embodiment, the calculating of a prediction rate may include determining, by the cloud server, a range of the minimum and maximum values of the user's biometric information that can be detected through the measurement device from the user's basic biometric characteristics, calculating, by the cloud server, the prediction rates of the trained prediction model corresponding to each biometric information included in the range of the minimum and maximum values of the biometric information, and transmitting, by the cloud server, some of the calculated prediction rates to the collection terminal as a caching table, wherein the outputting, by the cloud server or the collection terminal, of the prediction result includes detecting, by the collection terminal, any one prediction rate corresponding to biometric information received from the measurement device among prediction rates included in the caching table, and discriminating, by the collection terminal, a prediction result for the specific disease based on the detected prediction rate and a preset threshold, and outputting the discriminated prediction result.

### Advantageous Effects of Invention

In a health prediction system and a control method for the same according to the present disclosure, a user's health status may be diagnosed through a learning model trained according to the characteristics of a measurement device used by a user to measure biometric information for medical diagnosis and the user's biometric characteristics, thereby having an effect of providing a customized medical diagnosis service specialized for the user.

Furthermore, the present disclosure may detect a context around a user based on at least one other device and reflect different thresholds according to the detected user context to analyze a diagnosis result of a previously trained health prediction model, thereby dynamically determining whether a disease has developed according to the user's context. Therefore, in a case where a user is able to receive a medical service, whether a disease has developed may be more sensitively determined, thereby having an effect of allowing the user to respond more quickly to the occurrence of the disease and prepare for a disease occurrence context in advance.

In addition, the present disclosure may provide an authorized third party with only address information of a storage in which a user's medical information is stored other than the medical information previously stored therein, and only allows the viewing of the medical information through the address information, thereby having an effect of preventing the medical information from being transmitted to the third party and minimizing the leakage of medical information. Moreover, the address information may be distributively stored in a blockchain manner, thereby having an effect of strengthening the security of medical information according to the address information and preventing the leakage of the address information.

### BRIEF DESCRIPTION OF DRAWINGS

FIG. 1 is a block diagram showing a configuration of a health prediction system according to an embodiment of the present disclosure.

FIG. 2 is a block diagram showing a configuration of a server of a health prediction system according to an embodiment of the present disclosure.

FIG. 3 is a block diagram showing a configuration of a collection terminal that serves as a personal health gateway (PHG) of a health prediction system according to an embodiment of the present disclosure.

FIG. 4 is a block diagram showing a configuration of a biometric information measurement device or personal health device (PHD) of a health prediction system according to an embodiment of the present disclosure.

FIG. 5 is a flowchart showing an operation process of performing, by a health prediction system according to an embodiment of the present disclosure, training of a prediction model for diagnosing a user's health status according to the characteristics of a measurement device used by the user.

FIGS. 6A and 6B are exemplary diagrams for explaining the characteristics of a measurement device.

FIG. 7 is an exemplary diagram showing an example of a prediction model for diagnosing a user's health status in a health prediction system according to an embodiment of the present disclosure.

FIG. 8 is a flowchart showing an operation process of predicting a result in which whether a specific disease has developed is predicted by reflecting a user's context in a health prediction system according to an embodiment of the present disclosure.

FIG. 9 is an exemplary diagram showing an example in which a threshold is set differently depending on a user's context detected in a health prediction system according to an embodiment of the present disclosure.

FIG. 10 is a flowchart showing an operation process of training a variation in blood glucose level depending on a user's diet through a health prediction system according to an embodiment of the present disclosure.

FIG. 11 is a flowchart showing an operation process of providing a result in which a variation in blood glucose level depending on the user's diet is predicted through the prediction model trained in FIG. 10.

FIGS. 12A to 12C are conceptual diagrams for explaining methods of updating a prediction model in a health prediction system according to an embodiment of the present disclosure.

FIG. 13 is a flowchart showing an operation process of providing a caching table including diagnosis prediction results according to a user's biometric information in a health prediction system according to an embodiment of the present disclosure.

FIG. 14 is an exemplary diagram showing an example in which a caching interval for extracting the caching table of FIG. 13 is determined according to a user's biometric information and characteristics.

FIG. 15 is a flowchart showing an operation process of providing a diagnosis prediction result using a caching table in a health prediction system according to an embodiment of the present disclosure.

FIG. 16 is a flowchart showing an operation process for allowing an authorized third party to view a user's medical information in a health prediction system according to an embodiment of the present disclosure.

### MODE FOR THE INVENTION

It should be noted that technical terms used herein are merely used to describe specific embodiments, and are not intended to limit the present disclosure. Furthermore, a singular expression used herein includes a plural expression unless it is clearly construed in a different way in the context. A suffix “module” or “unit” used for elements disclosed in the following description is merely intended for easy description of the specification, and the suffix itself is not intended to have any special meaning or function.

As used herein, terms such as “comprise” or “include” should not be construed to necessarily include all elements or steps described herein, and should be construed not to include some elements or some steps thereof, or should be construed to further include additional elements or steps.

In addition, in describing technologies disclosed herein, when it is determined that a detailed description of known technologies related thereto may unnecessarily obscure the subject matter disclosed herein, the detailed description will be omitted.

Furthermore, the accompanying drawings are provided only for a better understanding of the embodiments disclosed herein and are not intended to limit technical concepts disclosed herein, and therefore, it should be understood that the accompanying drawings include all modifications, equivalents and substitutes within the concept and technical scope of the present disclosure. In addition, not only individual embodiments described below but also a combination of the embodiments may, of course, fall within the concept and technical scope of the present disclosure, as modifications, equivalents or substitutes included in the concept and technical scope of the present disclosure.

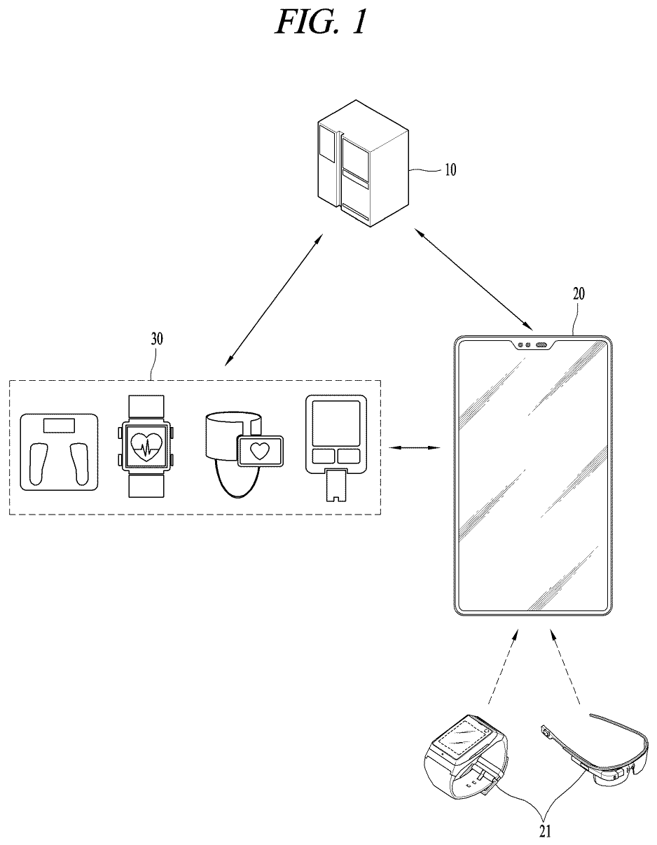

FIG. 1 is a block diagram showing a configuration of a health prediction system according to an embodiment of the present disclosure.

Referring to FIG. 1, a health prediction system according to an embodiment of the present disclosure may include at least one measurement device (PHD) 30 capable of measuring a user's biometric information, a collection terminal (PHG) 20 that collects and stores the user's biometric information measured by the at least one measurement device 30, and a server 10 that receives the measurement information stored in the collection terminal 20.

First, the measurement device 30 may be a device that measures the user's biometric information. The measurement devices may be classified according to the user's biometric information to be measured (e.g., blood pressure, weight, blood glucose level, heart rate, etc.). In addition, even if the biometric information to be measured is the same, it may be classified depending on the manufacturer, wearing method, or method of measuring biometric information.

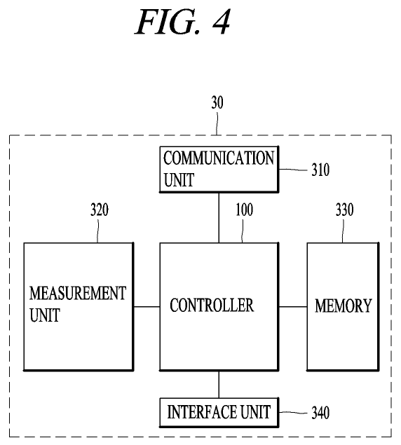

The measurement device 30 may automatically measure the user's biometric information according to the user's selection or at a preset cycle, and transmit the measured biometric information to the collection terminal 20 through a preset communication method. A more specific configuration of the measurement device will be described in more detail with reference to FIG. 4 below.

Furthermore, the collection terminal 20 may be connected to the at least one measurement device 30 through wireless communication. Furthermore, biometric information measured from each of the at least one measurement device 30 may be received and stored therein. In this case, the collection terminal 20 may request the measurement device to measure biometric information at a preset cycle, and the measurement device may measure the user's biometric information in response to the request and transmit the measured information to the collection terminal 20. That is, the collection terminal 20 may perform the role of a gateway, that is, a personal health gateway (PHG), between the server 10 and the measurement device 30, which stores information collected from at least one measurement device 30 and stores it in the server 10.

Meanwhile, the collection terminal 20 may be connected to the server 10 through wireless communication using a preset method. Additionally, measurement information collected from the at least one measurement device 30 may be transmitted to the server 10 according to a preset cycle or as soon as the measurement information is collected. Furthermore, in response to the transmitted measurement information, a diagnosis prediction result that has diagnosed the user's health status may be received. Additionally, the received diagnosis prediction result may be displayed so as to be identified by the user.

Meanwhile, a wireless communication method in which the collection terminal 20 is connected to the server 10 (hereinafter, referred to as a first communication method) and a wireless communication method in which the collection terminal 20 is connected to the at least one measurement device 30 (hereinafter, referred to as a second communication method) may be different from each other.

For example, the first communication method may be a communication method performed by transmitting and receiving wireless signals through a communication network based on wireless Internet technologies. In this case, examples of such wireless Internet access include Wireless LAN (WLAN), Wireless Fidelity (Wi-Fi), Wi-Fi Direct, Digital Living Network Alliance (DLNA), Wireless Broadband (WiBro), Worldwide Interoperability for Microwave Access (WiMAX), High Speed Downlink Packet Access (HSDPA), High Speed Uplink Packet Access (HSUPA), Long Term Evolution (LTE), LTE-Advanced (LTE-A), 5G communication, and the like.

In this case, the server 10 may constitute a communication network cloud network based on the wireless Internet technologies. Furthermore, the server 10 may be connected through the cloud network. In this case, the server 10 may be a cloud server.

Meanwhile, the second communication method may be a communication method based on short-distance communication technology. For example, the short-range communication technologies may include Bluetooth™, Radio Frequency Identification (RFID), Infrared Data Association (IrDA), Ultra Wideband (UWB), ZigBee, Near Field Communication (NFC), Wireless-Fidelity (Wi-Fi), Wi-Fi Direct, Wireless Universal Serial Bus (Wireless USB), and the like. More preferably, the second communication method may be a Bluetooth communication method, and in this case, a communication connection between the collection terminal 20 and at least one measurement device 30 may be established based on a Bluetooth Low Energy (BLE) generic ATTribute profile (GATT) protocol, which is a low-energy Bluetooth protocol.

Meanwhile, the collection terminal 20 may be a user's mobile terminal. In this case, the mobile terminal may include a mobile phone, a smart phone, a laptop computer, a digital broadcasting terminal, a personal digital assistant (PDA), a portable multimedia player (PMP), a navigator, a slate PC, a tablet PC, an ultrabook, a wearable device (e.g., a smart watch, smart glasses, a head mounted display (HMD)), or the like.

In addition, the collection terminal 20 may be connected to at least one nearby peripheral device in addition to the at least one measurement device 30. Furthermore, information sensed by the connected at least one peripheral device may be collected.

As an example, the peripheral device may be at least one wearable device 21 worn by the user. In this case, the user's location information or current time information collected by the wearable device may be collected by the collection terminal 20. Additionally, the peripheral device may be an Access Point (AP) disposed in a preset specific region. In this case, the AP may include information on a region where the device itself is disposed. Therefore, the collection terminal 20 may acquire information on a region in which the device is currently located based on the information collected from the AP.

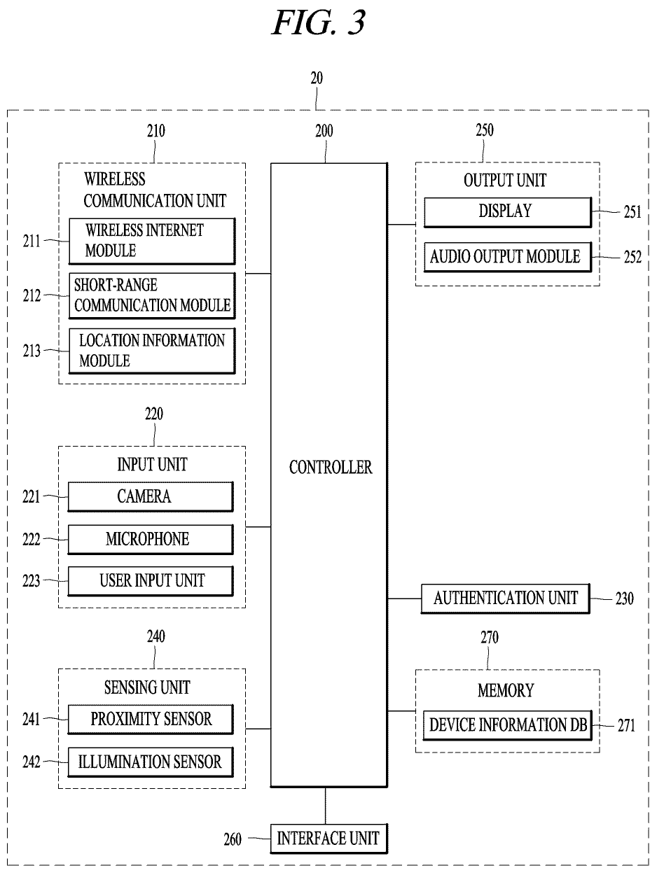

In this manner, information on peripheral devices collected by the collection terminal 20 may be stored as context information related to the user's current context. Then, the collection terminal 20 may infer the user's current context based on the collected context information. In addition, information on the inferred user's context may be transmitted to the server 10 together with biometric information measured by the at least one measurement device 30. A more specific configuration of the collection terminal 20 will be described in more detail with reference to FIG. 3 below.

The server 10 may be connected to the collection terminal 20 through a cloud network. Furthermore, the collected and stored information may be received from the collection terminal 20. Additionally, the user's health status may be diagnosed based on the information received from the collection terminal 20, and the diagnosed prediction result may be provided to the collection terminal 20 in response to the received information.

Here, the prediction result may be a prediction result for a specific disease or health status. As an example, the specific disease or health status may be diabetes or obesity. In this case, the server 10 may calculate a prediction rate of the user's diabetes or obesity based on the information received from the collection terminal 20, and predict a likelihood of developing diabetes or obesity based on the calculated prediction rate. Furthermore, the predicted result may be provided to the collection terminal 20 as the diagnosis prediction result.

In order to diagnose and predict such a health status, the server 10 may be provided with a prediction model that may predict a likelihood of developing the specific disease or health status based on biometric information to be input.

As an example, the prediction model may be configured in the form of an artificial neural network in which training is carried out using machine learning technology. In this case, the prediction model may include a plurality of hidden layers, and weights that are set to nodes, respectively, which constitute each of the hidden layers. Furthermore, weights set to respective nodes may be optimized through training that is carried out according to the machine learning technology.

Meanwhile, the server 10 may carry out the training of the prediction model by reflecting the characteristics of the measurement device 30 that is used by the user to acquire biometric information. As an example, the server 10 may change weights applied to respective hidden layers of an initial prediction model according to the characteristics of the measurement device 30 provided from the user. That is, as described above, when the measurement device 30 is classified according to the manufacturer, wearing method, or method of measuring biometric information, the server 10 may set different weights assigned to the respective hidden layers of the initial prediction model depending on the manufacturer, wearing method, or measurement method. To this end, the server 10 may request information on measurement devices that provide measurement information from the collection terminal 20, and the collection terminal 20 may transmit device characteristic information identified based on device identification information entered by the user or collected from the measurement device connected thereto, that is, information including the manufacturer, wearing method, or measurement method of the corresponding measurement device, to the server 10.

Meanwhile, the server 10 may classify input data according to the transmitted biometric information and input the classified data into the prediction model. For example, the server 10 may input biometric information measured from devices with different characteristics according to the device characteristic information as different groups of input information into the prediction model. Alternatively, the server 10 may input different groups of input information into the prediction model according to the kind of user's measured biometric information (e.g., blood pressure, weight, blood glucose level, heart rate, etc.). In this case, different groups of input information may be input into hidden layers at different levels or may be input into different nodes at the same level of hidden layers.

Meanwhile, the information transmitted from the collection terminal 20 may include not only the user's biometric information measured by the measurement device 30 but also information related to the user's context (context information).

Then, the server 10 may predict the user's health status based on a prediction rate calculated from the prediction model and the received context information. As an example, the server 10 may calculate a prediction rate of the user's diabetes or obesity based on the information received from the collection terminal 20, and predict a likelihood of developing a specific disease corresponding to the prediction rate calculated based on the context information.

In this case, the likelihood of developing a specific disease may be predicted by a true positive rate (TPR), which is a probability of being diagnosed as positive when there is a disease, a false positive rate (FPR), which is a probability of being diagnosed as positive when there is no disease, or true negative rate (TNR), which is a probability of being diagnosed as negative when there is no disease, and a false negative rate (FNR), which is a probability of being diagnosed as negative when there is a disease (in this case, TPR=1−FPR, TNR=1−FNR).

In this case, the true positive rate (TPR) or true negative rate (TNR) and the false positive rate (FPR) or false negative rate (FNR) may be determined based on a preset threshold. That is, as the threshold increases, the true positive rate or true negative rate may increase (a decrease in the false positive rate or false negative rate), and as the threshold decreases, the false positive rate or false negative rate may increase (a decrease in the true positive rate or true negative rate). Therefore, as the threshold decreases, a likelihood of outputting a prediction result that discriminates that there is a disease even when there is no disease may increase.

In this case, the server 10 may increase or decrease the threshold based on context information. As an example, when the user is in a hospital or the like, the server 10 may detect that the user is in the hospital through context information, and decrease the threshold. In this case, the false positive rate may increase, and thus it may be detected that the disease has developed according to a decreased threshold even if the prediction rate calculated based on the biometric information detected from the user does not actually reach a diseased level (a discrimination result based on a typical threshold). Therefore, even when the user's biometric information changes slightly, a prediction result indicating that the user has developed a disease may be transmitted, and the collection terminal 20 may output an alarm based on the prediction result. Therefore, whether a disease has developed may be predicted more sensitively based on the user's biometric information, which allows for a faster response when the disease has actually developed or prevents the development of the disease in advance.

Meanwhile, the server 10 may store the user's biometric information provided by the collection terminal 20. Furthermore, the address information of a storage area where the biometric information is stored may be provided to an authorized third party to allow the authorized third party to view the stored user's biometric information.

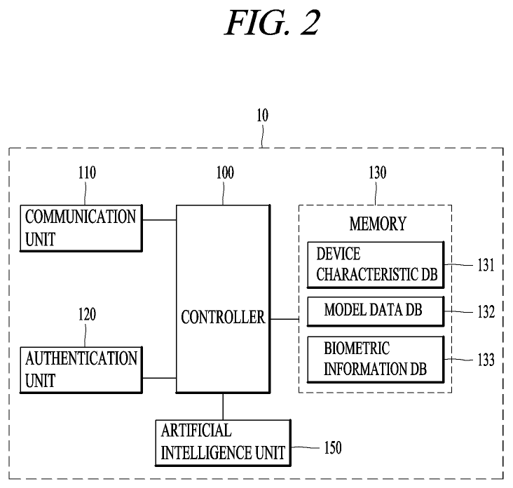

In this case, the address information may be distributively stored in a plurality of terminals constituting a blockchain network. In this case, the address information may be distributively stored as a plurality of data blocks. In addition, in the case of a blockchain algorithm, only those with tokens may access data blocks, so security may be strengthened. For this purpose, the server 10 may be connected to the blockchain network. A more specific configuration of the server 10 will be described in more detail with reference to FIG. 2 below.

Meanwhile, in the above description, an example in which the user's biometric information measured by at least one measurement device 30 is provided to the server 10 via the collection terminal 20 has been described. However, if the at least one measurement device 30 is provided with a communication module that allows a communication connection with the server 10, and if the measured biometric information can be stored in the measurement device, then the biometric information may, of course, be directly transmitted to the server 10 without going through the collection terminal 20. In this case, the collection terminal 20 may transmit only context information corresponding to the user's current context determined based on information collected from at least one peripheral device to the server 10.

FIG. 2 is a block diagram showing a configuration of the server 10 of a health prediction system according to an embodiment of the present disclosure.

Referring to FIG. 2, the server 10 of the health prediction system according to an embodiment of the present disclosure may include a controller 100, a communication unit 110 connected to the controller 100 and controlled by the controller 100, an authentication unit 120, a memory 130, and an artificial intelligence unit 150. The elements shown above in FIG. 2 are not essential for implementing the server 10, and thus the server 10 described herein may have more or fewer element than those listed above.

First, the communication unit 110 may include one or more modules that allow the server 10 to perform wireless communication with at least one other device using a preset communication technology. As an example, the communication unit 110 may include at least one wireless Internet module configured to transmit and receive wireless signals in a communication network based on WLAN, Wi-Fi, Wi-Fi Direct, DLNA, WiBro, WiMAX, HSDPA, HSUPA, LTE, LTE-A, 5G communication technologies, and the like. In addition, a communication network according to at least one of the wireless communication technologies may constitute a cloud network.

Furthermore, the authentication unit 120 may store and authenticate various information for authenticating an authority to view biometric information stored in the server 10. As an example, the authentication unit 120 may store an authorized third party's authentication key to authenticate the third party, and receive, when there is a requester to view biometric information, the requester's authentication key from the blockchain network and authenticate whether the requester is an authorized third party through the previously stored authentication key. Here, the third party's authentication key stored in the authentication unit 120 may be stored when the third party registers an account with the server 10.

Furthermore, when the requester is an authorized third party, a token allowing the requester to view the stored user's biometric information may be issued, and the issued token may be stored. In this case, the requester, that is, an authorized third party, may receive the address information of a storage area where the biometric information is stored through the token stored in the authentication unit 120, thereby viewing the user's biometric information stored in the server 10.

Meanwhile, the token may have an expiration time set, and may be automatically discarded when the set expiration time has elapsed. Therefore, even if he or she is an authorized third party, when the expiration time has elapsed, the user's biometric information may be viewed only by reissuing a token through an authentication process using the authentication key.

Meanwhile, the artificial intelligence unit 150, which performs a role of processing information based on an artificial intelligence technology, may include at least one module that performs at least one of learning of information, inference of information, perception of information, and processing of a natural language.

The artificial intelligence unit 150 may perform at least one of learning, inferring, and processing a vast amount of information (big data), such as information stored in the server 10, and information stored in an external storage capable of communicating with the server 10 using a machine learning technology.

Here, learning may be achieved through the machine learning technology. The machine learning technology is a technology that collects and learns a large amount of information based on at least one algorithm, and determines and predicts information on the basis of the learned information. The learning of information is an operation of grasping characteristics of information, rules and judgment criteria, quantifying a relation between information and information, and predicting new data using the quantified patterns.

Algorithms used by the machine learning technology may be algorithms based on statistics, for example, a decision tree that uses a tree structure type as a prediction model, an artificial neural network that mimics neural network structures and functions of living creatures, genetic programming based on biological evolutionary algorithms, clustering of distributing observed examples to a subset of clusters, a Montecarlo method of computing function values as probability using randomly-extracted random numbers, and the like.

As one field of the machine learning technology, deep learning is a technology of performing at least one of learning, determining, and processing information using the artificial neural network algorithm. The artificial neural network may have a structure of linking between layers (hidden layers), and transferring data between the layers. This deep learning technology may be employed to learn vast amounts of information through the artificial neural network using a central processing unit (CPU) optimized for parallel computing.

Meanwhile, in this specification, the artificial intelligence unit 150 and the controller 100 may be understood as the same element. In this case, functions performed by the controller 100 described herein may be expressed as being performed in the artificial intelligence unit 150, and the controller 100 may be referred to as the artificial intelligence unit 150, or conversely the artificial intelligence unit 150 may also be referred to as the controller 100.

On the other hand, in this specification, the artificial intelligence unit 150 and the controller 100 may be understood as separate elements. In this case, the artificial intelligence unit 150 and the controller 100 may perform various controls on the server 10 through data exchange with each other. The controller 100 may perform at least one of functions that are executable in the server 10, or control at least one of the elements of the server 10, based on a result derived from the artificial intelligence unit 150. Furthermore, the artificial intelligence unit 150 may also be operated under the control of the controller 100.

Meanwhile, the memory 130 stores data that support various functions of the server 10. The memory 130 may store a plurality of application programs (or applications) running on the server 10, data for operating the server 10, instructions, and data for operating the artificial intelligence unit 150 (e.g., at least one algorithm information for updating or training a machine learning or prediction model, etc.).

Additionally, information on at least one different biometric information measurement device 30 may be stored in the memory 130. For example, weight information corresponding to each characteristic of a different measurement device 30 and information on a hidden layer and node to which the weight is applied may be stored in the form of a database (DB) (device characteristic DB 131) in the memory 130. Accordingly, a different weight may be applied to a different hidden layer for each characteristic of the measurement device to generate a different initial prediction model for each measurement device used by the user, and training may be carried out through an initial prediction model reflecting the characteristics of the measurement device.

Here, the characteristics of the measurement device 30 may include a manufacturer of the measurement device 30, a method in which the measurement device is worn to measure biometric information, or a method in which the biometric information is measured. In this case, different weights to be applied according to the manufacturer, wearing method, measurement method, and the like, and at least one hidden layer to which the weights are applied may be determined.

In this case, a different weight and hidden layer may be determined for each characteristic of the measurement device 30. That is, the controller 100 of the server 10 may determine weights and hidden layers corresponding to the manufacturer of the input measurement device in a first order. Furthermore, other weights and hidden layers may be additionally determined depending on the wearing method of the measurement device to be input (in a second order). In addition, still other weights and hidden layers may be additionally determined depending on the measurement method of the measurement device to be input (in a third order). Then, the weights determined in the first to third orders may be applied to respectively determined hidden layers to generate an initial prediction model. Furthermore, the generated prediction model may be stored in a model data database (DB) 132.

In this case, if the user uses a plurality of measurement devices (e.g., first and second measurement devices) simultaneously, then the controller 100 of the server 10 may detect weights and hidden layers corresponding to the characteristics of the first and second measurement devices, respectively. Furthermore, the detected weights may be applied to the detected hidden layers to generate an initial prediction model customized to the device characteristics of the plurality of measurement devices. In this case, if conflicting weights are applied to a specific hidden layer, the weights applied to the corresponding hidden layer may, of course, conflict due to the application of the weights. Furthermore, the generated prediction model may be stored in the model data database (DB) 132.

To this end, the memory 130 of the server 10 may be provided with the device characteristic DB 131 in which various characteristic information of different measurement devices is stored in a database format. In addition, the model data DB 132 may be provided in which an initial prediction model generated according to the characteristics of the measurement device detected from the device characteristic DB 131 is stored. Here, the initial prediction model may be a prediction model that has not been trained, and training may be gradually carried out by the artificial intelligence unit 150.

Meanwhile, the memory 130 of the server 10 may further include a database (hereinafter, referred to as a biometric information DB 133) in which measured biometric information received through the communication unit 110 is stored. The biometric information DB 133 may store received biometric information in a classified manner by time stored or kind of biometric information to be stored, and store address information for an area on the memory 130 in which each of the classified biometric information is stored, for example, pointer information. In this case, the pointer information may be transmitted through the communication unit 110 under the control of the controller 100 of the server 10.

Meanwhile, the controller 100 may control each element connected thereto, and control an overall operation of the server 10.

When information on at least one measurement device 30 that measures the user's biometric information is received through the communication unit 110, the controller 100 may detect the information of weights and hidden layers according to the received characteristics of the measurement device 30 from the device characteristic DB 131. Furthermore, based on the detected information of weights and hidden layers, an initial prediction model customized for the measurement devices used by the user may be generated. In this case, the initial prediction model may be an artificial neural network model having a plurality of hidden layers each having a plurality of nodes. Furthermore, the generated initial prediction model may be stored as an initial prediction model in the model data DB 132.

Furthermore, when the user's biometric information measured by the measurement device 30 is received through the communication unit 110, the controller 100 may train the initial prediction model based on the received biometric information. Such training may be carried out until a preset training completion condition is satisfied.

As an example, the training completion condition may be satisfied through an accuracy test using a number of training data, a time at which training is carried out, or preset virtual biometric information.

Here, the accuracy test may be a test that compares a prediction rate calculated when the virtual biometric information whose prediction rate is known in advance is input into the prediction model with the previously known prediction rate. In this case, if the calculated prediction rate is within an error range from the prediction rate corresponding to the previously known virtual biometric information, then it may be determined that training is complete. However, otherwise, it may be determined that training is insufficient.

Meanwhile, when training is completed, the controller 100 may input the received biometric information into the trained prediction model, and compare a prediction rate output from the trained prediction model with a preset threshold. Furthermore, based on a threshold and a comparison result, a likelihood of developing or not developing a specific disease is predicted (positive prediction), and a result of predicting the likelihood of developing or not developing the disease (negative prediction) may be transmitted in response to the biometric information.

Meanwhile, the controller 100 may change the threshold based on the user's context information. In this case, as the threshold increases, the true positive rate or true negative rate may increase (a decrease in the false positive rate or false negative rate), and as the threshold decreases, the false positive rate or false negative rate may increase (a decrease in the true positive rate or true negative rate). Therefore, as the threshold decreases, a likelihood of outputting a prediction result that discriminates that there is a disease even when there is no disease may increase.

Therefore, as a result of discriminating the user's context based on the context information, when the user is in a hospital, or the like, a threshold may be set such that whether the disease has developed may be predicted more sensitively according to the user's biometric information. In this case, the controller 100 may further increase a false positive rate by decreasing the threshold. Therefore, even when the user's biometric information deteriorates slightly, it may be predicted that the disease has developed, and the prediction result may be transmitted to the collection terminal 20. Therefore, when the disease has actually developed, the user may respond more quickly, and prevent the development of the disease in advance by taking action prior to the development of the disease.

Meanwhile, the controller 100 may update the trained prediction model when a preset update condition is satisfied. For example, the update condition may be satisfied when a preset update cycle is expired (cycle-based update). Alternatively, it may be satisfied when a specific event occurs (event-based update). Here, the specific event may be a case where new measurement device information is received, or accuracy is lowered below a preset level as a result of its own accuracy test.

Here, the accuracy test may be the same or similar to an accuracy test for discriminating whether the training has been completed. As an example, the controller 100 may periodically perform its own accuracy test and determine whether to update the prediction model based on a result of performing the accuracy test.

The updating of the prediction model may be carried out in various ways. For example, at least part of respective hidden layers constituting a prediction model and at least part of weights that are set for respective nodes constituting the hidden layers may be changed to new values. In this case, since the weights that are set is the prediction model, this update method may correspond to replacing the prediction model with a new prediction model.

Here, the new values of the weights may be the weights of a new prediction model generated according to the characteristics of new measurement device information. Alternatively, they may be the weights of a prediction model generated during the process of training the prediction model. That is, they may be the weights of a prediction model prior to reflecting a recent training result.

Alternatively, the updating of the prediction model may be carried out through a combine or ensemble according to a plurality of prediction models including a currently trained prediction model.

As an example, the controller 100 may update the prediction model through a combine or ensemble between at least one new prediction model generated according to the characteristics of new measurement device information and a currently trained prediction model. Alternatively, the prediction model may be updated through a combine or ensemble between a plurality of prediction models that further include a prediction model generated in the process of training the prediction model. In this case, the prediction model generated in the process of training the prediction model may be a prediction model prior to reflecting a recent training result.

Meanwhile, the updated prediction model may be trained on the received biometric information until a preset training completion condition is satisfied. In this case, the controller 100 may provide a prediction result depending on the prediction rate and threshold calculated according to the previously trained prediction model in response to the received biometric information until the training of the updated prediction model is completed. Furthermore, after the training of the updated prediction model is completed, the prediction result according to the prediction rate and threshold calculated according to the updated prediction model may be provided in response to the received biometric information.

Meanwhile, when there is a request for address information indicating the address of an area where the user's specific biometric information is stored from the collection terminal 20 through the communication unit 110, the controller 100 may transmit the address information. Here, the specific biometric information may be a specific kind of biometric information stored at a specific time. Then, the address information may be transmitted to an authorized third party's terminal through the collection terminal 20. Furthermore, when a request for biometric information corresponding to the address information is received from the authorized third party's terminal, the controller 100 may cause the third party's terminal to retrieve the user's specific biometric information stored in the storage area corresponding to the address information.

Meanwhile, FIG. 3 is a block diagram showing a configuration of the collection terminal 20 that serves as a personal health gateway (PHG) of a health prediction system according to an embodiment of the present disclosure.

The collection terminal 20 may include a wireless communication unit 210, an input unit 220, an authentication unit 230, a sensing unit 240, an output unit 250, an interface unit 260, a memory 270, and a controller 200. The elements shown in FIG. 3 are not essential for implementing the collection terminal 20, and thus the collection terminal 20 described herein may have more or fewer element than those listed above.

In more detail, among the elements, the wireless communication unit 210 may include one or more modules that allow wireless communication between the collection terminal 20 and a wireless communication system, between the collection terminal 20 and at least peripheral device, or between the collection terminal 20 and an external server.

The wireless communication unit 210 may include at least one of a wireless Internet module 211, a short-range communication module 212, a location information module 213 and the like.

The wireless Internet module 211 refers to a module for wireless Internet access, and may be internally or externally coupled to the collection terminal 20. The wireless Internet module 211 may transmit and receive wireless signals via communication networks according to wireless Internet technologies.

The wireless Internet technologies may include WLAN, Wi-Fi, Wi-Fi Direct, DLNA, WiBro, WiMAX, HSDPA, HSUPA, LTE, LTE-A, 5G communication technologies, and the like, and the wireless Internet module 211 may transmit and receive data according to at least one wireless Internet technology in a range including Internet technologies not listed above.

The short-range communication module 212, which is for short-range communication, may support short-distance communication using at least one of Bluetooth, RFID, infrared communication, UWB, ZigBee, NFC, Wi-Fi, Wi-Fi Direct, and wireless USB technologies. The short-range communication module 212 may support wireless communication between the collection terminal 20 and a wireless communication system, between the collection terminal 20 and a peripheral device, or between the collection terminal 20 and a network in which an external server is located through wireless area networks.

Here, the peripheral device may be a wearable device (e.g., a smart watch, smart glasses, a head mounted display (HMD), etc.) capable of exchanging data with (or interoperating with) the collection terminal 20 according to the present disclosure. The short-range communication module 212 may detect (recognize) a wearable device capable of communicating with the collection terminal 20 around the collection terminal 20.

The location information module 213 is a module for acquiring the location (or current location) of the collection terminal 20, and a representative example thereof is a Global Positioning System (GPS) module or a Wireless Fidelity (WiFi) module. For example, when the collection terminal utilizes a GPS module, the location of the collection terminal 20 may be acquired using signals sent from GPS satellites. As another example, when the collection terminal 20 utilizes a Wi-Fi module, the location of the collection terminal 20 may be acquired based on information from the Wi-Fi module and a wireless access point (AP) that transmits or receives wireless signals. If necessary, the location information module 213 may replaceably or additionally perform any one function of the other modules of the wireless communication unit 210 to obtain data with regard to the location of the collection terminal 20. The location information module 213 is a module used to acquire the location (or current location) of the collection terminal 20, and is not limited to a module that directly calculates or acquires the location of the collection terminal 20.

The input unit 220 may include a camera 221 for receiving an image signal, a microphone 222 or an audio input unit for receiving an audio signal, or a user input unit 223 (e.g., a touch key, a push key (or a mechanical key), etc.) for receiving information from the user. Speech data or image data collected by the input unit 220 may be analyzed and processed by a user's control command.

Furthermore, the authentication unit 230 may store and authenticate various information for authenticating an authority to view biometric information stored in the server 10. As an example, the authentication unit 230 may store address information transmitted from the server 10 in response to a request from an authorized third party. The address information may be pointer information indicating address information corresponding to an area of the memory 130 of the server 10 in which the user's specific biometric information is stored.

In addition, the authentication part may store identification information to identify the user and authenticate an authority for use, and may include a user identity module (UIM), a subscriber identity module (SIM), a universal subscriber identity module (USIM), and the like.

The sensing unit 240 may include one or more sensors for sensing at least one of information within the collection terminal 20, information on surrounding environment information around the collection terminal 20, and user information. For example, the sensing unit 240 may include a proximity sensor 241, an illumination sensor 242, a touch sensor, an acceleration sensor, a magnetic sensor, a G-sensor, a gyroscope sensor, a motion sensor, an RGB sensor, an infrared (IR) sensor, a ultrasonic sensor, an optical sensor (e.g., refer to the camera 221), a microphone 222, a battery gage, an environment sensor (e.g., a barometer, a hygrometer, a thermometer, a radiation detection sensor, a thermal sensor, a gas sensor, etc.), and a chemical sensor (e.g., an electronic nose, a health care sensor, a biometric sensor, etc.). Meanwhile, the collection terminal 20 disclosed herein may utilize information sensed by at least two of the sensors by combining them.

The output unit 250 is intended to generate an output related to visual, auditory, or tactile senses, and may include a display 251 and an audio output unit 252. The display module 251 may have an inter-layered structure or an integrated structure with a touch sensor in order to implement a touch screen. The touch screen may provide an output interface between the collection terminal 20 and the user, as well as functioning as the user input unit 223 that provides an input interface between the collection terminal 20 and the user.

The interface unit 260 may serve as an interface with various types of external devices connected to the collection terminal 20. The interface unit 260, for example, may include wired or wireless headset ports, external power supply ports, wired or wireless data ports, memory card ports, ports for connecting a device having an identification module, audio input/output (I/O) ports, video I/O ports, earphone ports, or the like. The collection terminal 20 may perform an appropriate control associated with a connected external device, in response to the external device being connected to the interface unit 260.

In addition, the memory 270 stores data that support various functions of the collection terminal 20. The memory 270 may store a plurality of application programs (or applications) running on the collection terminal 20, data or instructions for operating the collection terminal 20. At least some of those application programs may be downloaded from an external server via wireless communication. In addition, at least some of these applications may be present on the collection terminal 20 from the time of shipment for the basic functions of the collection terminal 20 (e.g., a measurement information collection and transmission function, an output of a received prediction result). Meanwhile, the application programs may be stored in the memory 270, installed in the collection terminal 20, and executed by the controller 200 to perform an operation (or function) of the collection terminal 20.

Meanwhile, the memory 270 may store information on different measurement devices that may be connected to the collection terminal 20. For example, the memory 270 may store identification information on measurement devices manufactured by a plurality of different manufacturers and information on the characteristics of respective measurement devices corresponding to the identification information. Here, the characteristics of the measurement device may include manufacturer information, a kind of biometric information measured by the measurement device, and a type of the measurement device (e.g., in the case of a blood pressure monitor, an arm-type or watch-type). In addition, the characteristics of the measurement device may include a method in which the measurement device is mounted on the user to measure biometric information (hereinafter, referred to as a mounting method, e.g. a contact type, an attachment type, a pressure type, etc.), and a method in which the measurement device measures biometric information from the user (hereinafter, referred to as measurement method, e.g. an invasive or non-invasive type, a continuous or single measurement type, etc.).

Therefore, the collection terminal 20 may identify, when a measurement device is connected thereto, various characteristic information of the measurement device through identifying the connected measurement device. Furthermore, the characteristic information of the identified measurement device may be transmitted to the server 10. Accordingly, the server 10 may determine a hidden layer and weights to be set for nodes of the hidden layer from the device characteristic DB 131 based on the characteristic information of the measurement device, and generate an initial prediction model corresponding to the received characteristic of the measurement device.

Meanwhile, if the measurement device is not identified based on the identification information received from the measurement device, or if identification information is not received from the measurement device, then the controller 200 of the collection terminal 20 may directly receive the characteristic information of the currently connected measurement device from the user. In this case, the user may directly input measurement device characteristic information including at least one of a manufacturer of the measurement device, a kind of biometric information being measured, a type of the measurement device, a mounting method, and a measuring method. Then, the collection terminal 20 may transmit the collected measurement device characteristic information to the server 10.

Meanwhile, the controller 200 may typically control an overall operation of the collection terminal 20 in addition to the operations associated with the application programs. The controller 200 may provide or process information or functions appropriate for a user in a manner of processing signals, data, information and the like, which are input or output through the above-described elements, or activating the application programs stored in the memory 170.

Furthermore, the controller 200 may control at least part of the elements illustrated in FIG. 3, to execute an application program stored in the memory 270. Moreover, the controller 200 may operate at least two of the elements included in the collection terminal 20 in combination with each other in order to execute the application program.

Meanwhile, the controller 200 may perform a communication connection to at least one measurement device 30. Here, the at least one measurement device 30 may be connected through the short-range communication module 212. More preferably, the at least one measurement device may be connected using a Bluetooth Low Energy (BLE) communication method. Furthermore, the user's biometric information received from the connected at least one measurement device 30 may be received, and the received biometric information may be transmitted to an external server, that is, the server 10, which is connected through the wireless Internet module 211. Furthermore, in response to the transmitted biometric information, a diagnosis prediction result for the user's health status, that is, a prediction result for the likelihood of developing at least one preset disease, may be received from the server 10, and the received prediction result may be output through at least one of the display 251 and the audio output unit 252.

In addition, the controller 200 may perform a communication connection to at least one peripheral device 21. Furthermore, the user's context may be analyzed based on at least one peripheral device connected thereto, and a detection result of the sensing unit 240, or the location information of the location information module 213. Additionally, information on the analyzed context, that is, context information, may be transmitted to the server 10 separately from the measured biometric information. Then, the server 10 may perform a diagnosis prediction for the user's health status based on the received context information.

In addition, the controller 200 may store, when the address information of a storage area where the user's specific biometric information is stored is received from the server 10 in response to a request to view biometric information from an authorized third party, that is, a third party with a token that allows the viewing of the user's biometric information, the received address information. Furthermore, when a request for the address information is received from the authorized third party, the stored address information may be transmitted to the third party's terminal in response to the request.

Meanwhile, the controller 200 may process biometric information received from at least one measurement device 30 into information in a form required by the server 10. As an example, the controller 200 may convert biometric information received from at least one measurement device 30 into information in a format required by the server 10. Alternatively, the controller 200 may transmit secondary biometric information calculated from primary biometric information received from at least one measurement device 30 to the server 10.

As an example, the controller 200 may calculate the user's oxygen saturation and calorie consumption based on a step count and movement received from biometric information that is received from at least one measurement device 30. In addition, information such as the calculated oxygen saturation and calorie consumption may be transmitted to the server 10 as biometric information received from the at least one measurement device 30. Then, the server 10 may input the secondary biometric information into a previously trained prediction model. That is, the controller 200 may convert the biometric information received from the at least one measurement device or calculate other biometric information from the biometric information received from the at least one measurement device to process the biometric information collected from the at least one measurement device 30 into a form of biometric information that can be input into a previously trained prediction model. Furthermore, the processed biometric information may be transmitted to the server 10.

FIG. 4 is a block diagram showing a configuration of the biometric information measurement device 30 of a health prediction system according to an embodiment of the present disclosure.

Referring to FIG. 4, in a health prediction system according to an embodiment of the present disclosure, the measurement device 30 that measures the user's biometric information may include a controller 300, a communication unit 310 connected to the controller 300 and controlled by the controller 300, a measurement unit 320, a memory 330, and an interface unit 340. In addition, although not shown, it may further include a display and an audio output part. The elements shown in FIG. 4 are not essential for implementing the measurement device 30, and thus the measurement device 30 described herein may have more or fewer element than those listed above.

First, the communication unit 310 may include at least one communication module for wireless communication with the collection terminal 20. As an example, the communication unit 310 may include at least one module to which short-range communication technology is applied. More preferably, the communication unit 310 may be a communication module for Bluetooth Low Energy (BLE) communication.

Meanwhile, the communication unit 310 may further include a wireless Internet module that allows wireless Internet access. In this case, by using the wireless Internet module, the measurement device 30 may be directly connected to the server 10 without going through the collection terminal 20 to transmit the user's biometric information measured through the measured device 30 to the server 10.

The measurement unit 320 may include at least one sensor for detecting a specific kind of user biometric information. As an example, the measurement unit 320 may include a blood glucose sensor that detects the user's blood glucose level using an invasive measurement method or the like, or a sensor such as a blood pressure sensor, a pulse sensor, or a body temperature sensor that measures a body temperature. Alternatively, when the measurement device 30 is a weight scale or a height gauge, it may include a weight sensor for detecting the user's weight or a length sensor for detecting the user's height.

The characteristics of the measurement device may be determined depending on the type of sensor provided in the measurement unit 320. For example, a measurement method (for example, an invasive method) in which the measurement device 30 measures the user's biometric information may be determined depending on the sensor provided in the measurement device 30. In addition, a wearing method in which the measurement device 30 is worn by the user may be determined according to a method of fixing the sensor to the user for the measurement.

The memory 330 stores data that supports the functions of measurement device 30. The memory 330 may store an application program running on the measurement device 30, data or instructions for operating the measurement device 30. Additionally, a measurement result of the measurement unit 320 may be stored therein.

The interface unit 340 serves as an interface with various types of external devices connected to the measurement device 30. The controller 300 may perform appropriate control related to an external device connected thereto, in response to the external device being connected to the interface unit 340.

Furthermore, the controller 300 typically controls an overall operation of the measurement device 30. The controller 300 may process signals, data, information, and the like, which are input or output through the above-described elements or run an application program stored in the memory 330, thereby displaying information measured by the measurement unit 320 or providing the measured information to the server 10 or the collection terminal 20.

As an example, the controller 300 may control the measurement unit 320 according to a preset cycle to detect the user's biometric information. Alternatively, when there is a request from the collection terminal 20, the measurement unit 320 may be controlled to detect the user's biometric information. Furthermore, the detected biometric information may be transmitted to the collection terminal 20. Alternatively, when direct wireless communication with the server 10 is allowed, the measured biometric information may be directly transmitted to the server 10.

Hereinafter, embodiments related to a control method that can be implemented in the health prediction system including the server 10, the collection terminal 20, and the measurement device 30 will be described with reference to the accompanying drawings. It is obvious to those skilled in the art that the present disclosure can be embodied in other specific forms without departing from the concept and essential characteristics thereof.

In the following description, it will be described on the assumption that the server 10, which is a server 10 that can be connected through a cloud network, for the sake of convenience, is a cloud server 10.

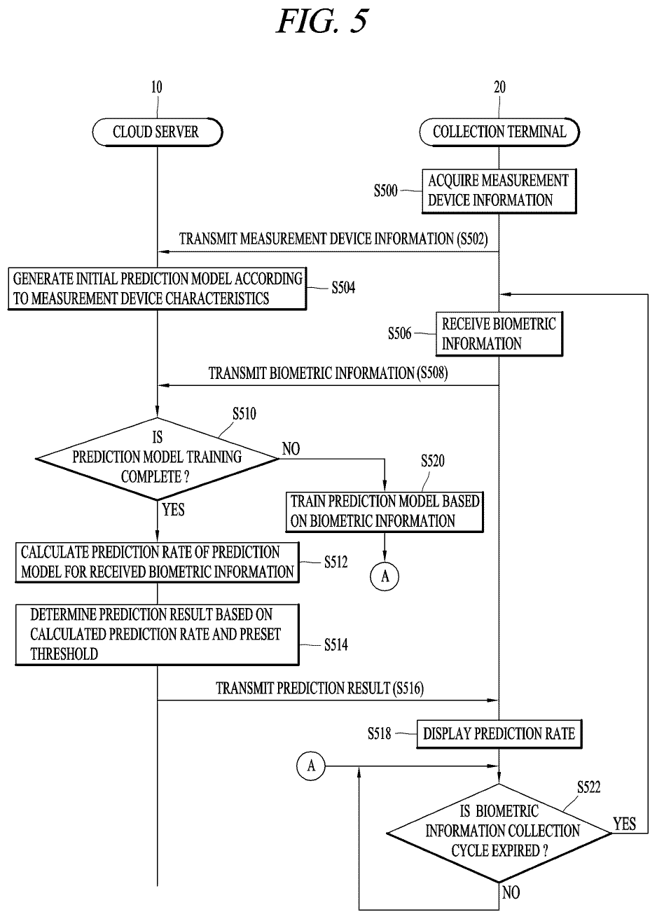

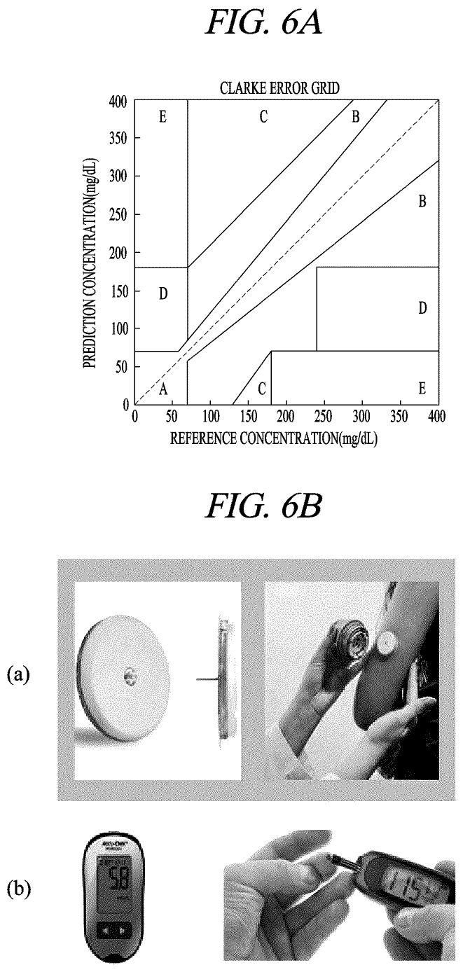

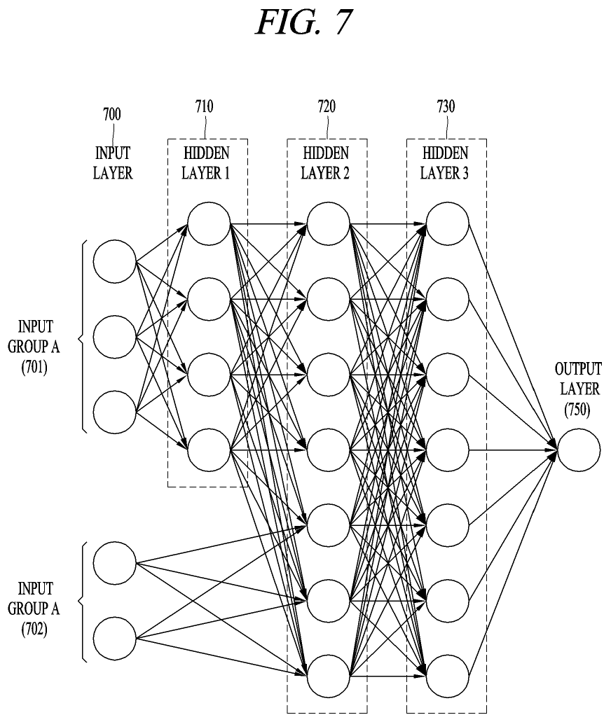

FIG. 5 is a flowchart showing an operation process of performing, by a health prediction system according to an embodiment of the present disclosure, training of a prediction model for diagnosing a user's health status according to the characteristics of a measurement device used by the user. Furthermore, FIGS. 6A and 6B are exemplary diagrams for explaining the characteristics of a measurement device. In addition, FIG. 7 is an exemplary diagram showing an example of a prediction model for diagnosing a user's health status in a health prediction system according to an embodiment of the present disclosure.

First, referring to FIG. 5, the collection terminal 20 of the health prediction system according to an embodiment of the present disclosure may first transmit information on at least one measurement device 30 connected thereto to the cloud server 10. Here, the information on the measurement device 30 may be identification information of the measurement device 30, and may be information on the characteristics of the measurement device 30.

As an example, the collection terminal 20 may detect, when the measurement device 30 is connected thereto, the characteristic information of the measurement device from a device information DB 271 based on the identification information received from the connected measurement device to acquire the characteristic information of the measurement device. Alternatively, the collection terminal 20 may acquire the characteristic information of the connected measurement device based on the user's input regarding the connected measurement device (S500). Furthermore, the acquired measurement device characteristic information may be transmitted to the cloud server 10 (S502).

Here, the characteristic information of the measurement device may include characteristics that quantify the clinical accuracy of the measurement device, that is, quantified clinical accuracy. As an example, when the measurement device is a blood glucose meter, it may include characteristic information according to a Clarke Error Grid as shown in FIG. 6A.

The Clark Error Grid is intended to quantify the clinical accuracy of a patient's estimate of a current blood glucose level compared to a blood glucose level obtained from a measuring meter, wherein the Clark Error Grid characteristic of a measurement device is characteristic information that quantifies the clinical accuracy of a blood glucose estimate detected by the measurement device with respect to a reference level, and each area represents an error from a measurement value of a reference sensor (area A), an area that is outside an error range but does not lead to inappropriate treatment (area B), an area that leads to unnecessary treatment (area C), an area that fails to detect hypoglycemia or hypertension (area D), and an area that confuses hypoglycemia treatment with hyperglycemia treatment (area E). Furthermore, a state in which clinical test results of blood glucose estimates from a specific measurement device (blood glucose meter) are distributed in each area of the Clark Error Grid may represent the quantified clinical accuracy of the measurement device. Furthermore, the quantified clinical accuracy of the measurement device (blood glucose meter) analyzed through the Clark Error Grid may be detected as the characteristic information of the blood glucose meter.

Meanwhile, the characteristic information of the measurement device may include information on the wearing method and measurement method of the measurement device.

For example, in the case of a blood glucose meter, there may be an attachment type as shown in (a) of FIG. 6B and a contact type as shown in (b) of FIG. 6B. In this case, the blood glucose meters shown in (a) and (b) of FIG. 6B may have different characteristic information regarding the wearing method. On the contrary, as shown in (a) and (b) of FIG. 6B, blood glucose meters that measure blood glucose level in an invasive manner may have the same characteristic information regarding the measurement method.

Meanwhile, the cloud server 10 that has received the characteristic information of the measurement device from the collection terminal 20 may detect the weights of hidden layers and nodes corresponding to each of the received characteristic information of the measurement device from the device characteristic DB 131. Furthermore, based on the detected weights of hidden layers and nodes, an initial prediction model reflecting the received characteristic information of the measurement device may be generated (S504).

Here, the initial prediction model may be a model for predicting a likelihood of developing at least one specific disease. Furthermore, the at least one specific disease (hereinafter, referred to as a specific disease) may be a disease corresponding to the biometric information acquired in the step S504. That is, a prediction model for predicting a likelihood of developing a specific disease may be determined based on biometric information measured by a measurement device. In this case, the prediction model may be a model that can predict the likelihood of developing the at least one disease based on biometric information to be input.

Alternatively, the specific disease may be a disease directly selected by the user. For example, when there are multiple diseases related to the biometric information acquired in the step S504, a prediction model for predicting a likelihood of developing at least one disease selected by the user among the diseases may be determined.

Then, the cloud server 10 may set the weights of hidden layers and nodes corresponding to each characteristic information of the measurement device for the determined prediction model for a specific disease, thereby generating an initial prediction model reflecting the characteristic information of the measurement device (S504).

The initial prediction model may be an artificial neural network model including a plurality of hidden layers and weights assigned to nodes included in each of the plurality of hidden layers. The artificial neural network model may be a model in which the number of hidden layers passing therethrough is designed to be different depending on input information that is input through an input layer. FIG. 7 shows an example of an artificial neural network model (prediction model) according to an embodiment of the present disclosure.

Referring to FIG. 7, a prediction model of a health prediction system according to an embodiment of the present disclosure may include at least one exclusive layer 710 and at least one common layer 720, 730. Here, the exclusive layer 710 may be a hidden layer that includes weights applied only to inputs classified into a specific group. In addition, the common layer 720, 730 may be a hidden layer that includes weights applied to inputs of all groups.

That is, as shown in FIG. 7, when inputs classified into group A are applied thereto, weights according to the exclusive layer 710 may be assigned via the exclusive layer 710. Furthermore, values to which the weights of respective nodes that are set in the exclusive layer 710 are applied may be input to the common layer 720, 730. That is, both the weights assigned by the exclusive layer 710 and the weights assigned by the common layer 720, 730 may be applied to the inputs classified into the group A.

On the contrary, when inputs classified into group B are applied thereto, they may be input directly to the common layer 720, 730 without going through the exclusive layer 710. That is, to inputs classified into the group B, only the weights assigned by the common layer, 720, 730 may be applied without the weights assigned by the exclusive layer 710 being applied.

Here, input information that is input to the prediction model may be biometric information. Furthermore, a group of the input information may be a characteristic of a measurement device that has measured the biometric information. That is, the cloud server 10 may classify biometric information received from the measurement device into a specific group according to the received characteristics of the measurement device, and set or not set the exclusive layer 710 corresponding to the specific group. Therefore, biometric information received from a measurement device classified into a specific group may be input to the common layer 720, 730 via the exclusive layer 710 in which weights corresponding to the specific group are set, and thus a prediction model reflecting the characteristics of the measurement device collected from the collection terminal 20 may be generated.

Meanwhile, the collection terminal 20 may receive biometric information detected by a measurement device from the measurement device connected thereto (S506). Furthermore, the received biometric information may be transmitted to the cloud server 10 (S508).

Then, the cloud server 10 may discriminate whether the training of the initial prediction model generated in the step S504 has been completed (S510). Here, whether the training has been completed may be discriminated in various ways. For example, the cloud server 10 may discriminate whether training has been completed depending on whether the training has been carried out on a sufficient number of training data (biometric information). Alternatively, the cloud server 10 may discriminate whether training has been completed based on a period during which the training has been carried out. Alternatively, the cloud server 10 may discriminate whether the training has been completed through an accuracy test using biometric information whose prediction rate is known in advance. In this case, the cloud server 10 may input biometric information whose prediction rate is known in advance into a prediction model currently being trained, and discriminate whether training has been completed based on an error between a prediction rate calculated from the prediction model being trained and the prediction rate that is known in advance.

If training is not completed as a result of discriminating whether training has been completed in the step S510, then the cloud server 10 may perform training for the prediction model based on the currently received biometric information (S520). Then, the collection terminal 20 may discriminate whether a preset biometric information collection cycle has expired (S522), and if the biometric information collection cycle has expired, then the collection terminal 20 may proceed to step S506 again to receive biometric information measured by the measurement device from the measurement device. In this case, the process from step S506 to step S510 may be performed again.

On the contrary, when it is determined that the training of the prediction model is completed as a result of the discrimination in the step S510, the cloud server 10 may input the biometric information received in the step S504 into the trained prediction model. Furthermore, a prediction rate corresponding to the biometric information may be calculated through the trained prediction model (S512).

Meanwhile, as described above, the prediction model may be a prediction model for predicting a likelihood of developing a specific disease. Therefore, a prediction rate calculated from the prediction model may be a likelihood of developing a specific disease. Then, the cloud server 10 may discriminate a prediction result for the specific disease, that is, whether the disease has developed, based on the likelihood of developing the disease. In this case, the cloud server 10 may predict whether the specific disease has developed based on a preset threshold, and whether the prediction rate exceeds the threshold (S514). Furthermore, the predicted result, that is, whether the specific disease has developed, may be transmitted to the collection terminal 20 (S516).

Then, the collection terminal 20 may output the received prediction result, that is, whether a specific disease has developed, through the output unit 250 (S518). In this case, the collection terminal 20 may output whether the disease has developed through at least one of visual information displayed through the display 251 and audio information output through the audio output unit 252.

Meanwhile, the collection terminal 20 may discriminate whether a preset biometric information collection cycle has expired (S522), and if the biometric information collection cycle has expired, then the collection terminal 20 may proceed to the step S506 of receiving biometric information from the measurement device again. Therefore, the process from step S506 to step S518 may be performed again.

Meanwhile, the cloud server 10 of the health prediction system according to an embodiment of the present disclosure may discriminate a threshold for discriminating whether the specific disease has developed based on the user's context. In this case, when the threshold is different, whether the specific disease has developed may be determined to be different even if the prediction rate is the same. That is, in a case where the prediction rate calculated through the trained prediction model is 50%, if the threshold is 30%, then the cloud server 10 may discriminate that the user has developed a specific disease and transmit the prediction result to the collection terminal 20. On the contrary, if the threshold is 70%, then the cloud server 10 may discriminate that the user does not develop a specific disease and transmit the prediction result to the collection terminal 20.

The cloud server 10 may vary the threshold based on the user's context. For example, if urgent action is required even when the user's health status deteriorates even slightly, such as when the user is hospitalized, then the cloud server 10 may set the threshold lower than usual. Then, even when the prediction rate is lower than the usual case, the prediction result may be displayed as if the specific disease has developed, so the user may quickly receive treatment for the specific disease. That is, it may be possible to more sensitively detect the deterioration of the user's health status, and accordingly, the user may receive more urgent medical treatment according to the deterioration of the health status, or receive medical services to respond to the worsening health status before the specific disease actually develops, thereby preventing the development of the specific disease in advance.

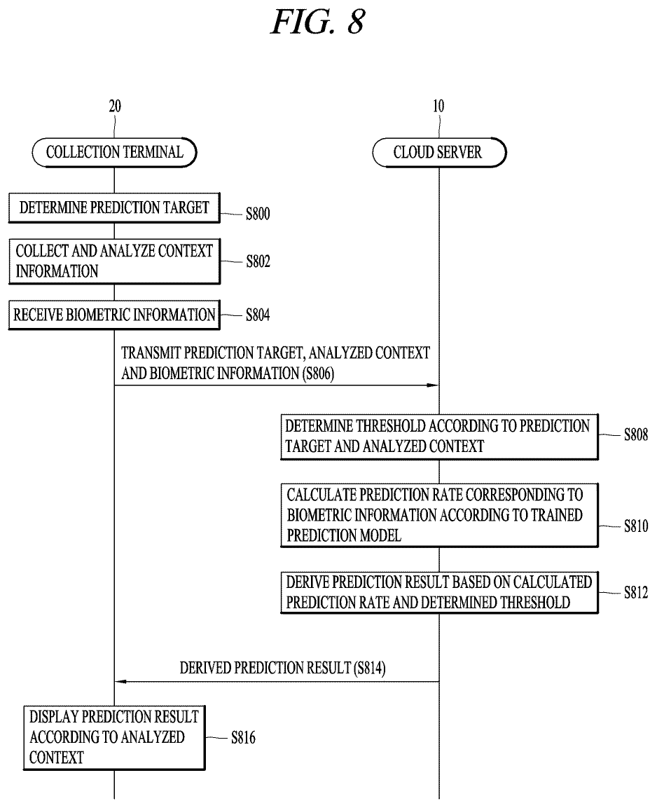

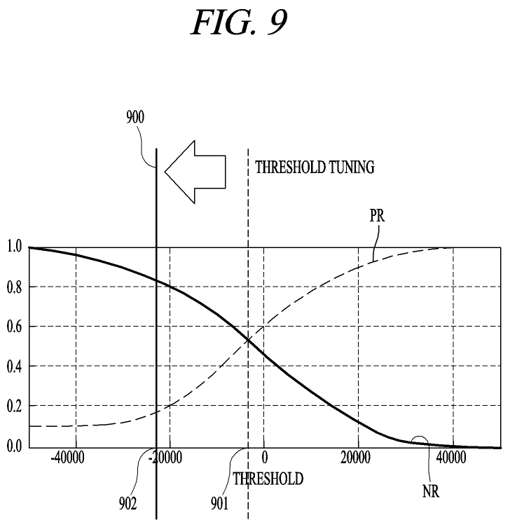

FIG. 8 is a flowchart showing an operation process of predicting a result in which whether a specific disease has occurred is predicted by reflecting a user's context in a health prediction system according to an embodiment of the present disclosure as described above. In addition, FIG. 9 is an exemplary diagram showing an example in which a threshold is set differently depending on a user's context detected in a health prediction system according to an embodiment of the present disclosure.

First, referring to FIG. 8, the collection terminal 20 may determine a prediction target for predicting whether a disease has developed (S800). Here, the prediction target may be a specific disease or specific condition. Furthermore, an initial threshold may be determined according to the determined prediction target.

The prediction target may be directly selected by the user. For example, when the user inputs a specific disease through the collection terminal 20, a prediction target may be determined as the specific disease. Alternatively, the prediction target may be automatically set to a specific disease corresponding to a currently set prediction model.

Meanwhile, according to the above description, the specific disease may be provided in plurality. That is, the prediction model trained in the cloud server 10 may be a model that can predict a likelihood of developing a plurality of diseases based on biometric information to be input. In this case, a different threshold may be set for each disease, and whether the each disease has developed may be determined based on the different threshold set for each disease and a prediction rate calculated from the prediction model. In this case, the prediction target may refer to any one disease among a plurality of diseases that can be predicted by the prediction model.

If the prediction target is determined in the step S600, then the collection terminal 20 may analyze the user's current context (S602). To this end, the collection terminal 20 may be connected to at least one peripheral device, and collect information related to the user's context from peripheral devices connected thereto.

For example, the collection terminal 20 may collect at least one item of location information related to its own location. For example, the collection terminal 20 may detect a location calculated from the location information module 213. Alternatively, the collection terminal 20 may collect information on a current location from an AP installed in a region where the terminal itself is located. Furthermore, the user's context may be inferred based on the collected location information.

Alternatively, the collection terminal 20 may collect information on at least one mobile terminal located nearby. For example, the collection terminal 20 may compare the identification information of at least one mobile terminal located around the collection terminal 20 with the identification information of mobile terminals of pre-registered acquaintances. Furthermore, if at least one of the identification information of the pre-registered acquaintances is located nearby, then the user's context may be inferred based on the at least one pre-registered acquaintance. As an example, the collection terminal 20 may infer that the user is located around family members based on the identification information of mobile terminals of family members located around the user.

Meanwhile, when the user's surrounding context and location are detected in this manner, the collection terminal 20 may analyze whether the user is in a preset context. For example, the collection terminal 20 may discriminate whether the user is in a hospital as a result of analyzing the user's location. Furthermore, when the user is located in a hospital, it may be detected that the user is in the preset context. In this manner, when it is discriminated that the user is in a preset context, the collection terminal 20 may generate information on the user's context, that is, context information.

Meanwhile, the collection terminal 20 may receive the user's biometric information measured by a connected measurement device according to a preset cycle or at the user's request, separately from information on the user's surrounding context (S804). Furthermore, when biometric information is received in step S804, the received biometric information, the context information, and information on a currently selected prediction target may be transmitted to the cloud server 10 (S806).

Then, the cloud server 10 may first input the received biometric information into the trained prediction model to calculate a prediction rate. Furthermore, among thresholds corresponding to diseases, respectively, that can be predicted by the prediction model, the threshold of a disease corresponding to the prediction target may be determined.

Meanwhile, the cloud server 10 may change the determined threshold based on the context information received from the collection terminal 20 (S808). For example, when the user's context corresponding to the context information received from the collection terminal 20 is a preset context (e.g., when the user is in a hospital), the cloud server 10 may change the threshold of a disease corresponding to the prediction target by a preset displacement. To this end, the memory 130 of the cloud server 10 may include context information corresponding to a plurality of different contexts and information on a variation displacement of a different threshold corresponding to each of the plurality of context information. As an example, when the user is in a general ward, in an emergency ward, and at home, the variation displacement of the threshold for each context may be different.

Meanwhile, when the threshold is determined according to the prediction target and the analyzed context in the step S808, the cloud server 10 may calculate a prediction rate corresponding to the received biometric information according to the trained prediction model (S810). Furthermore, from the calculated prediction rate, a prediction result of whether a disease to be predicted has developed may be derived based on a threshold determined according to the user's current context and the prediction target in the step S808 (S812). Furthermore, the derived prediction result may be transmitted to the collection terminal 20 (S814). Then, the collection terminal 20 may output the received prediction result through the output unit 250 (S816).

FIG. 9 is a diagram showing a correlation between a positive prediction rate (positive rate (PR)) and a negative prediction rate (negative rate (NR)) determined according to each prediction rate and threshold when the prediction rate is 0 (0%) to 1.0 (100%).

Referring to FIG. 9, it can be seen that when the positive prediction rate increases, the negative prediction rate decreases, and conversely, when the positive prediction rate decreases, the negative prediction rate increases. Here, the positive prediction rate is a prediction rate indicating that the user has developed a disease to be predicted, and the negative prediction rate is a prediction rate indicating that the user has not developed the disease to be predicted. That is, the positive prediction rate and negative prediction rate may have conflicting values.

Furthermore, when a specific threshold is set, a positive prediction rate reference and a negative prediction rate reference corresponding to the set threshold may be determined. That is, as the threshold increases, a positive prediction rate reference, that is, a reference value of a prediction rate that discriminates whether the disease to be predicted has developed, may increase, and conversely, a reference value of a prediction rate (negative prediction rate) that discriminates whether the disease to be predicted has not developed may decrease. Conversely, as the threshold decreases, a reference value of a prediction rate (positive prediction rate) that discriminates whether a disease to be predicted has developed may decrease, and conversely, a reference value of a prediction rate (negative prediction rate) that discriminates whether the disease to be predicted has not developed may decrease.

In this state, when the user is located in a hospital, the cloud server 10 may reduce the threshold by a preset variation displacement. Therefore, as shown in FIG. 9, a threshold 900 may be changed from a first value 901 to a second value 902.

When the threshold 900 is changed in this manner, a reference value of a positive prediction rate corresponding to the changed threshold 900 may decrease. Conversely, a reference value of a negative prediction rate corresponding to the changed threshold 900 may increase. Therefore, when a prediction rate is calculated from a trained prediction model, a probability of being determined to be positive, that is, a likelihood of being determined that a disease has developed, may further increase, and a likelihood of being determined that a disease has not developed may further decrease. Therefore, according to the prediction result determined through the received biometric information, it may be more easily discriminated that the user has developed a disease corresponding to the prediction target.

Meanwhile, when it is determined that a disease corresponding to the prediction target has developed, since the user is in a hospital, the user may receive medical services for the disease to be predicted. Therefore, when the user is located in a hospital, or the like, he or she may more sensitively receive medical services for the disease to be predicted according to a change in health status based on biometric information. In addition, even if it is a prediction rate that is not determined to have developed a disease in normal cases, it may be discriminated to have developed the disease by changing the threshold to receive medical services. Therefore, the present disclosure has an effect of preventing the development of the disease to be predicted in advance by allowing the user to receive medical services prior to the development of the disease to be predicted.

Meanwhile, in the case of a health prediction system according to an embodiment of the present disclosure, a prediction model trained from the user may be used, and thus the user may, of course, input virtual information to simulate in advance a change in the user's health status according to the input virtual information.

For example, if the health prediction system according to an embodiment of the present disclosure predicts the user's health status based on a blood glucose level detected through a blood glucose meter, then the user may enter information on food that has not yet been consumed, thereby predicting a variation in the user's blood glucose level and providing the predicted result to the user.

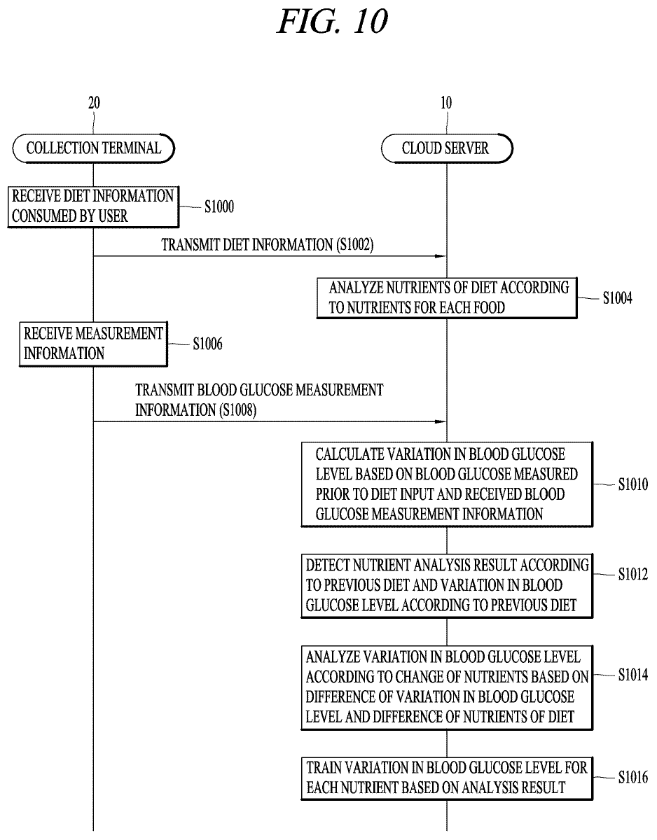

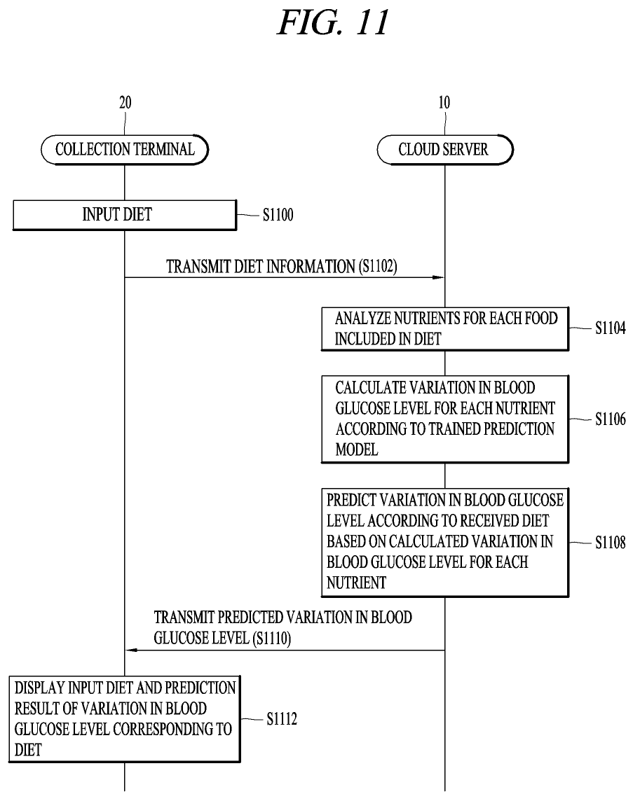

FIGS. 10 and 11 show these examples.

First, referring to FIG. 10, FIG. 10 is a flowchart showing an operation process of training a variation in blood glucose level depending on a user's diet through a health prediction system according to an embodiment of the present disclosure.

In this case, the collection terminal 20 may receive information on a diet consumed by the user from the user (S1000). Furthermore, the information on the diet entered by the user may be transmitted to the cloud server 10 (S1002). Here, the diet may include information on a name of at least one food and an intake amount of the food (e.g., weight or portion, for example, 1 serving, 1.5 servings, 2 servings).

Then, the cloud server 10 may analyze the nutrients of each food included in the received diet information. Furthermore, based on the analyzed nutrients, the nutrients corresponding to the entered diet and the amount of each nutrient consumed by the user may be analyzed (S1004).

In the step S1004, the cloud server 10 may detect nutrients corresponding to each kind of food included in the diet. To this end, the memory 130 of the cloud server 10 may include nutritional analysis information for each of a plurality of different foods, that is, information on the nutrients (e.g. carbohydrates, proteins, . . . ) contained in each food and the composition ratio of each nutrient relative to the reference weight.

Furthermore, the cloud server 10 may calculate an amount of each nutrient consumed by the user based on the intake amount of each food. For example, if the intake amount entered by the user is weight, then a ratio difference between the reference weight and the intake weight may be reflected in a nutritional analysis result of each food to calculate an amount of nutrients contained in each food consumed by the user. In addition, if the intake amount entered by the user is a portion (intake portion), then a weight of food corresponding to the intake portion may be calculated based on a weight of the reference portion (1 serving), an amount of nutrients contained in each food consumed by the user may be calculated based on a ratio difference between the calculated weight of the food and the reference weight. Furthermore, by summing the intake amounts of nutrients from each food, the nutrients consumed by the user and the amount of those nutrients for the entire diet may be calculated.

Meanwhile, when the user consumes food and a preset period of time elapses, the measurement device may measure the user's blood glucose level. Furthermore, the blood glucose level measured by the measurement device may be transmitted to the collection terminal 20, and the collection terminal 20 may receive it (S1006). Then, the collection terminal 20 may transmit the received blood glucose measurement information to the cloud server 10 (S1008).

The cloud server 10 that has received the blood glucose measurement information may compare a previously received blood glucose measurement result, that is, a blood glucose measurement result measured before the user consumes food, with a blood glucose measurement result received in the step S1008 to calculate a variation in blood glucose level (S1010). In this case, the variation in blood glucose level may be a variation in blood glucose level relative to a diet currently consumed by the user.

Furthermore, the cloud server 10 may detect the nutrients of a previously collected diet and a variation in blood glucose level measured for the diet (S1012). Furthermore, based on a variation in nutrients and blood glucose level in the previous diet detected in the step S1012 and a variation in nutrients and blood glucose level in a diet currently consumed by the user, which is measured in the step S1010, a variation in blood glucose level according to a change in nutrients may be calculated (S1014). That is, the cloud server 10 may detect a difference between nutrients in a previous diet and nutrients in a diet currently consumed by the user, and detect a difference between a variation in blood glucose level detected for the nutrients in the previous diet and a variation in blood glucose level detected for the nutrients in the diet currently consumed by the user. Accordingly, it may be possible to detect a difference in blood glucose level variation for the difference between the nutrients consumed by the user.

Then, the cloud server 10 may train a blood glucose level variation prediction model for each nutrient based on a difference in blood glucose level variation according to the calculated difference in nutrients (S1016).

The training process may be repeated a preset number of times or more, and when the repetition is completed the preset number of times or more, the cloud server 10 may determine that the training of the blood glucose level variation prediction model for each nutrient has been completed. Alternatively, the cloud server 10 may determine that, for at least one nutrient, when the magnitude of a blood glucose level variation changed according to a training result of the step S1016 is less than a preset level, the training of the blood glucose level variation prediction model for each nutrient has been completed. That is, in a case where it is difficult to expect the effect of training any longer, the cloud server 10 may determine that the training of the blood glucose level variation prediction model for each nutrient has been completed.

Meanwhile, FIG. 11 is a flowchart showing an operation process of providing a result of predicting a variation in blood glucose level according to a virtual diet entered by the user through the prediction model trained in FIG. 10, that is, a blood glucose level variation prediction model for each nutrient.

Referring to FIG. 11, the user may input virtual diet information through the collection terminal 20 according to an embodiment of the present disclosure (S1100). In this case, the virtual diet information, which is information on a diet that is not consumed by the user, may be information on a diet including foods to be consumed by the user in the future. Furthermore, the collection terminal 20 may transmit virtual diet information entered by the user to the cloud server 10 (S1102). Here, the virtual diet information may include information on a virtual intake amount.

Meanwhile, the cloud server 10 may analyze the nutrients of each food included in the received virtual diet (S1104). In this case, the nutrients for each food may be analyzed, and the intake amount of each nutrient for each food may be analyzed based on the virtual intake amount.

Then, the cloud server 10 may calculate a variation in blood glucose level corresponding to each nutrient through the blood glucose level variation prediction model for each nutrient trained in FIG. 10 (S1106). In this case, the variation in blood glucose level calculated through the trained blood glucose level variation prediction model for each nutrient may be a variation in blood glucose level according to the reference weight of each nutrient. Accordingly, the cloud server 10 may calculate a variation in blood glucose level corresponding to the intake amount of the corresponding nutrient according to the calculated variation in blood glucose level according to the reference weight of each nutrient and the intake amount of the corresponding nutrient analyzed in the step S1104.

Furthermore, for each nutrient, the cloud server 10 may add up a variation in blood glucose level corresponding to an intake amount of the nutrient. Furthermore, the added-up result may be predicted as a variation in blood glucose level corresponding to a virtual diet received in the step S1102 (S1108).

Furthermore, the predicted variation in blood glucose level may be provided to the collection terminal 20 in response to the virtual diet information received in the step S1102 (S1110). Then, the collection terminal 20 may output the received information on the predicted variation in blood glucose level through the output unit 250 (S1112). In this case, information on the received virtual diet and information on the predicted variation in blood glucose level may be displayed together on the display 251.

Meanwhile, the health prediction system according to an embodiment of the present disclosure may recommend an optimal diet to the user based on the trained blood glucose level variation prediction model for each nutrient. In this case, the trained blood glucose level variation prediction model for each nutrient may be the user's blood glucose level variation characteristics for each nutrient trained according to the user's blood glucose level measurement result. Therefore, the cloud server 10 may recommend an optimal diet based on the trained user's blood glucose level variation characteristics for each nutrient.

As an example, the cloud server 10 may detect at least one other user with similar biological characteristics to the user and recommend the user's diet based on diet information entered by the detected at least one other user (collaborate filtering method). That is, the cloud server 10 may compare each user's blood glucose level variation prediction model for each nutrient, and recommend the user's diet based on diet information entered by at least one other user who has a similar blood glucose level variation prediction model for each nutrient.

In this case, when there is a diet similar to the user's taste among the diet information entered by at least one other user, the cloud server 10 may recommend the diet information to the user (content based filtering method). In this case, whether tastes are similar to each other may be determined when the kinds of foods included in the diet match at a preset ratio or more.

Meanwhile, when a recommended diet is determined based on diet information entered by at least one other user, the cloud server 10 may provide information on the recommended diet to the collection terminal 20. In this case, the cloud server 10 may provide each food constituting the recommended diet, a result of predicting a blood glucose level variation for each food calculated according to a nutrient analysis result of the food, and glycemic index (GI) information corresponding to each food.

Here, the glycemic index information (GI), which is a value that compares a degree of increase in blood glucose level after consuming a predetermined amount of the corresponding food with that in blood glucose level after consuming the same amount of standard carbohydrate food, may refer to an index indicating a degree of increase in blood glucose level of the corresponding food for the same amount of standard carbohydrate content.

[Table 1] below shows an example of recommended diet information provided from the cloud server 10.

[symbol]

### Table 1

Meanwhile, the cloud server 10 of the health prediction system according to an embodiment of the present disclosure may perform an update on the trained prediction model when a preset update condition is satisfied.

In this case, the preset update condition may be satisfied when a preset update cycle is expired. Alternatively, the preset update condition may be satisfied when a measurement device is added, changed, or removed.

Alternatively, the update condition may be satisfied when accuracy is out of an error range as a result of testing the accuracy based on preset biometric information whose prediction level is known in advance. This is because the accuracy of a trained prediction model may decrease over time.

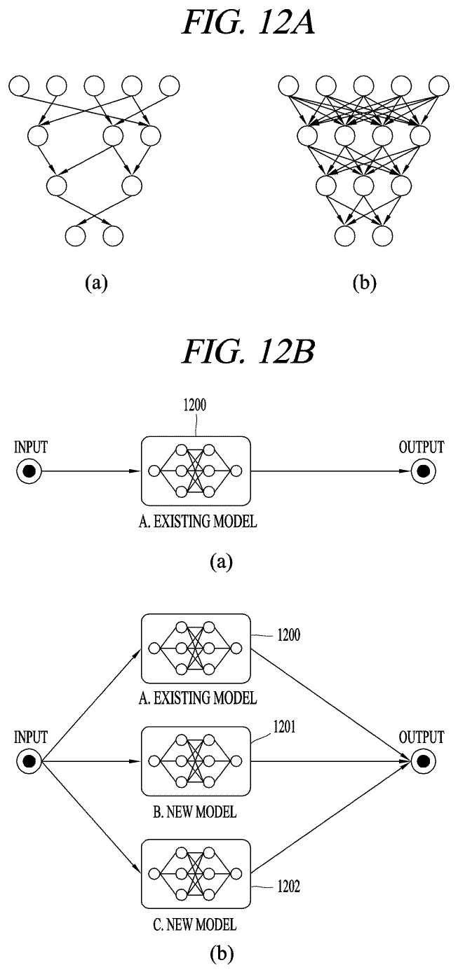

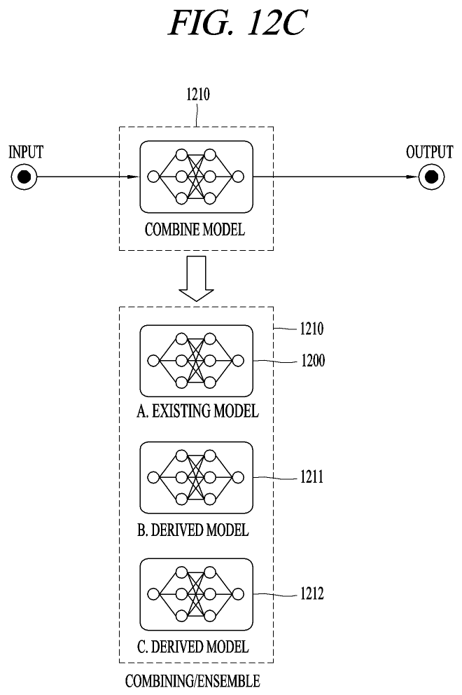

FIGS. 12A to 12C are conceptual diagrams for explaining methods of updating a prediction model in a health prediction system according to an embodiment of the present disclosure as described above.

First, referring to FIG. 12A, (a) of FIG. 12A shows an example of a prediction model prior to being updated, and (b) of FIG. 12A shows an example of a prediction model subsequent to being updated. As shown in FIG. 12A, the cloud server 10 may change at least some of the weights of at least some hidden layers of a currently trained prediction model.

In this case, since the weights set for the nodes of the hidden layers are the prediction model, in the case of FIG. 12A, at least part of the currently trained prediction model may be replaced according to new data. In this case, the new data may be data based on the characteristics of a newly added or changed measurement device.

That is, when a new measurement device is added or changed, a new hidden layer, for example, at least one exclusive layer or common layer, may be added as shown in (a) and (b) of FIG. 12A. Alternatively, at least one exclusive layer or common layer may be changed to another layer. In this case, the other layer may be a layer in which a weight assigned to at least some of the nodes constituting the layer is different.

On the contrary, when the measurement device is removed, some exclusive layers or common layers constituting the currently trained prediction model may be removed. In this case, the model as shown in (b) of FIG. 12A may be changed to a model as shown in (a) of FIG. 12A through update.

Meanwhile, when the prediction model is updated by replacing, adding, or removing at least some of the hidden layers, the cloud server 10 may perform retraining on the updated prediction model. To this end, the cloud server 10 and the collection terminal 20 may repeatedly perform the process from steps S506 to S522 of FIG. 5.

Meanwhile, unlike the case of FIG. 12A, the cloud server 10 may further generate a new prediction model without changing the existing prediction model, and use the existing model and the new model in combination.

For example, as shown in (a) of FIG. 12B, when calculating a prediction rate using one prediction model (model A 1200) prior to updating, if the event condition is satisfied, then the cloud server 10 may further generate at least one new model that meets the event condition.

As an example, when a new measurement device is added and an existing measurement device is replaced with another device, the cloud server 10 may generate a new first new model (model B 1201) according to the device characteristics of the newly added measurement device and a second new model (model C 1202) according to the device characteristics of the replaced measurement device.

Here, the first and second new models 1201, 1202 may be prediction models specialized for specific input information. That is, it may be a prediction model specialized for a specific measurement device, or a prediction model specialized for a specific kind of biometric information, a specific biometric information measurement method, a specific measurement device wearing method, or a specific measurement device from a specific manufacturer.

Meanwhile, the cloud server 10 may input bio-signals measured from the user to the existing model 1200, the first new model 1201, and the second new model 1202, respectively. Furthermore, prediction rates output from respective prediction models 1200, 1201, 1202 may be merged.

In this case, the cloud server 10 may calculate a statistical value for prediction rates calculated from respective prediction models 1200, 1201, 1202. As an example, the statistical value may be one of an average value, a maximum value, and a minimum value. Furthermore, the calculated statistical value may be transmitted to the collection terminal 20 as a prediction rate calculated for the received biometric information.

Here, the cloud server 10 may assign different weight ratios to a prediction rate calculated from the existing prediction model 1200, and respective prediction rates calculated from the first and second new models 1201, 1202, respectively. For example, the weight ratio may be set to 50% for the prediction rate of the existing prediction model 1200, and the weight ratio may be set to 25% for the prediction rate of the first new model 1201. In addition, the weight ratio may be set to 25% for the prediction rate of the second new model 1202. Furthermore, a prediction rate corresponding to the input biometric information may be calculated by adding up prediction rates according to the weights.

In this case, the weight ratio may be determined according to the training state of each model. Here, the first and second new models 1201, 1202 may not have completed training. Therefore, a lower weight ratio may be set for the prediction rates of the first and second new models 1201, 1202 for which training has not been completed compared to the existing model 1200 for which training has been completed. Then, the cloud server 10 may simultaneously perform training on the first and second new models 1201, 1202.

Meanwhile, if the first and second new models 1201 and 1202 are models specialized for specific input information, then the weight ratio of a specific new model may increase depending on input information provided from the collection terminal 20, that is, biometric information (or information from a measurement device). As an example, if the first new model 1201 is a specialized model that is specialized for the currently received biometric information, then the cloud server 10 may set the weight ratio of the existing model 1200 and the second new model 1202 to 0%. Furthermore, the weight ratio of the first new model 1201 may be set to 100%. In this case, the prediction rate of the first new model 1201 may be transmitted to the collection terminal 20 as a prediction rate corresponding to the received biometric information.

Meanwhile, when multiple prediction models are used as shown in FIG. 12B, a calculation time of the prediction rate may be delayed. Therefore, the cloud server 10 may limit in advance the maximum number of usable models. Furthermore, when performing an update of a prediction model, if the number of prediction models exceeds the limited maximum number of usable models, then the prediction models may be removed in the order of the oldest elapsed time subsequent to being generated. Accordingly, it may be possible to prevent a prediction rate from taking too much time to calculate.

Meanwhile, unlike using a plurality of prediction models in combination as shown in FIG. 12B, the cloud server 10 may, of course, update a prediction model by combining a plurality of prediction models with one another.

In this case, as shown in FIG. 12C, the cloud server 10 may generate an existing model 1200 and at least one model (a first derived model 1211, a second derived model 1212) derived from the training process of the existing model 1200 as a model to be combined therewith. That is, the first derived model 1211 and the second derived model 1212 may be the existing model 1200 prior to the completion of training.

In this case, the cloud server 10 may overlap or combine the existing model 1200 with at least part of the first derived model 1211 and the second derived model 1212. That is, for example, nodes with the same weight in each hidden layer may be maintained, and nodes with different weights may be added to each hidden layer. In this case, as described above, the first derived model 1211 or the second derived model 1212, which is the existing model 1200 prior to the completion of training, and may be a prediction model the number of hidden layers including an exclusive layer or common layer is the same.

The cloud server 10 may generate a combine model or an ensemble model (hereinafter, referred to as a combine model 1210) by overlapping and merging at least some of the plurality of prediction models. Then, training may be performed again on the updated prediction model using the generated combine model 1210. Therefore, the cloud server 10 and the collection terminal 20 may repeatedly perform the process from steps S506 to S522 of FIG. 5.

Meanwhile, as described above, when a prediction result is calculated for each bio-signal received from the collection terminal 20 and the calculated prediction result is provided to the collection terminal 20, the time it takes for the prediction result to be transmitted subsequent to transmitting biometric information may increase. In addition, when there is a large number of biometric information received, an amount of computation of the cloud server 10 may increase, which may lead to a computational load of the cloud server 10.

Accordingly, when the training of the prediction model is completed, the health prediction system according to an embodiment of the present disclosure may calculate in advance prediction rates corresponding to bio-signals corresponding to a predetermined range, and transmit in advance the calculated prediction rates to the collection terminal 20, thereby reducing the amount of computation of the cloud server 10, and shortening a time required until a prediction result corresponding to the bio-signal is transmitted.

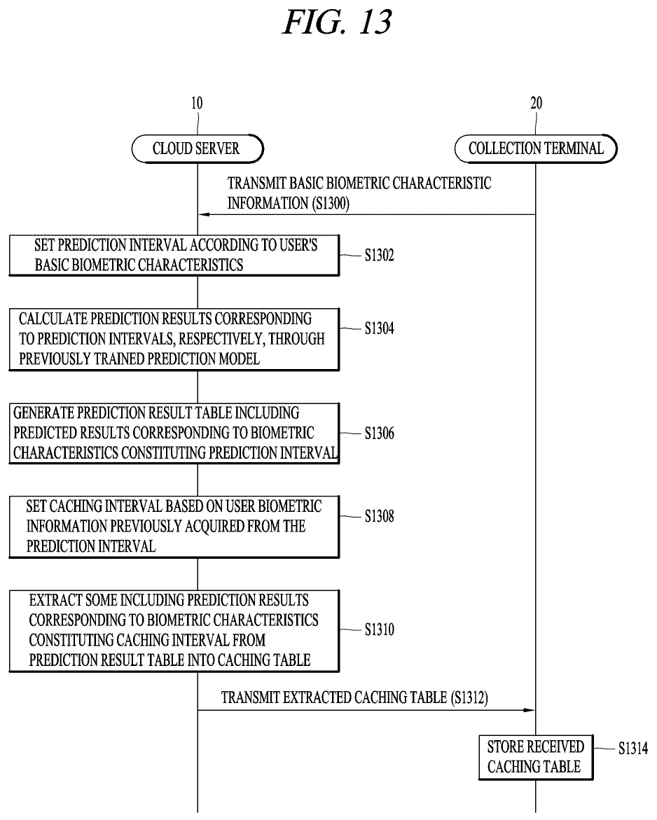

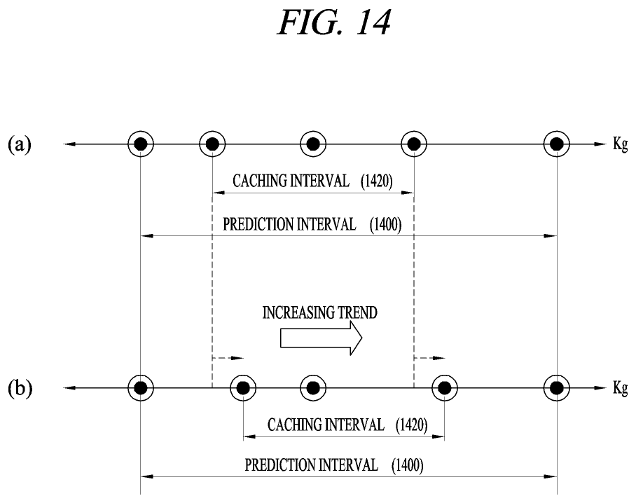

FIG. 13 is a flowchart showing an operation process of providing, by the cloud server 10, a caching table including diagnosis prediction results according to a user's biometric information to the collection terminal 20 in a health prediction system according to an embodiment of the present disclosure as described above. Furthermore, FIG. 14 is an exemplary diagram showing an example in which a caching interval for extracting the caching table of FIG. 13 is determined according to a user's biometric information and characteristics.

Referring to FIG. 13, the collection terminal 20 may transmit information on the user's basic biometric characteristics (S1300). Here, the user's basic biometric characteristic information, which is information on basic user's universal biometric characteristics, such as the user's gender and age group, may be information stored in the collection terminal 20 or collected from the user's information registered with the measurement device.

Then, the collection terminal 20 may set a universal prediction interval corresponding to the information on the received basic biometric characteristics (S1302). For example, when the user is a man with a universal body type, the universal prediction interval for body weight may be set from 20 to 220 kg. Furthermore, when the user is a woman, she usually weighs less than a man, so a universal prediction interval (e.g., 20 to 150 kg) may be set in a smaller range than the universal prediction interval corresponding to the man.

In addition, in this manner, 50 to 250 cm may be set as a universal prediction interval for the user's height.

The universal prediction interval may be an arbitrary range of biometric information that may be maximized in a typical case with respect to the received user's basic biometric characteristics. The universal prediction interval may be determined according to a number of experimental or statistical results related to the present disclosure. In the following description, for the sake of convenience of explanation, assuming that the biometric information detected from the user is weight and height, an example in which a prediction rate for diabetes disease is detected based on the weight and height will be described.

When a universal prediction interval is set from the user's basic biometric characteristics in the step S1302, the cloud server 10 may calculate in advance prediction results corresponding to the universal prediction intervals, respectively, through a prediction model in which a likelihood of developing diabetes disease is trained according to the user's height and weight (S1304). For example, the cloud server 10 may set virtual heights corresponding to a height interval in the universal prediction interval for each preset unit (e.g., 1 cm), and set virtual weights corresponding to a weight range of the universal prediction interval for each preset unit (e.g., 1 kg). Furthermore, by inputting virtual weights corresponding to virtual heights, respectively, into the prediction model, prediction results corresponding to each biometric information (a height for each interval and a weight for each interval) constituting the universal prediction interval may be calculated.

In this case, if a height range set as a universal prediction interval is 50 to 250 cm, and a weight range set as the universal prediction interval is 20 to 220 kg, and if the basic unit of height is 1 cm and the basic unit of weight is 1 kg, then a total of 40,000 prediction results may be calculated.

Meanwhile, when prediction results corresponding to the virtual biometric information, respectively, constituting the universal prediction interval are calculated, the cloud server 10 may generate a prediction result table including the calculated prediction results (S1306). Therefore, a prediction result table including diabetes disease prediction rates matching a plurality of items of biometric information, respectively, constituting the universal prediction interval may be generated as shown in [Table 2] below.

[symbol]

### Table 2

Then, the cloud server 10 may set part of the universal prediction interval as a caching interval based on the previously acquired user's biometric information (S1308). For example, when the user's height according to the previously acquired user's biometric information is 160 cm, the cloud server 10 may set a predetermined range as a caching interval based on the previously acquired user's height of 160 cm. In this case, if the predetermined range is 20 cm, then a caching interval may be set for heights between 140 cm and 180 cm.

Meanwhile, when the user's weight according to the previously acquired user's biometric information is 60 kg, the cloud server 10 may set a predetermined range as a caching interval based on the previously acquired user's weight of 60 kg. In this case, if the predetermined range is 20 kg, then a caching interval may be set for weights between 40 kg and 80 kg.

Then, the cloud server 10 may extract an area corresponding to the caching interval set in the step S1308 into a caching table from the prediction result table (S1310). Therefore, a portion of a prediction interval table corresponding to a portion (140 to 180 cm) of a universal prediction interval of any one kind of biometric information (height) constituting a prediction interval table and a portion (40 to 80 kg) of the universal prediction intervals of another kind of biometric information (weight) constituting the prediction interval table may be extracted into a caching table. In this case, the caching table may be a table including diabetes disease prediction rates corresponding to the user's biometric characteristics (height and weight) in the extracted each caching interval, as shown in [Table 3] below.

[symbol]

### Table 3

Then, the cloud server 10 may transmit the caching table extracted in step the S1310 to the collection terminal 20 (S1312). Then, the collection terminal 20 may store the received caching table (S1314).

Meanwhile, the caching interval may be dynamically set based on a directionality according to the user's biological characteristics. For example, if the user's age is in a teenager range, the user's weight and height may be in an increasing trend. On the contrary, when the user's age is in a middle-age range, the user's weight may be in an increasing trend, but the user's height may be in a stationary trend. Based on the directionality of each user's age, the cloud server 10 may dynamically set the caching interval.

FIG. 14 shows an example in which the caching interval is dynamically set according to the user's biological characteristics (e.g., age group).

As described above, when the user's age group is in a teenage range, the user's weight and height may be in an increasing trend compared to now. Therefore, the cloud server 10 may set an interval increased by a predetermined value from the caching interval set according to previously acquired biometric information of the user as the caching interval.

Referring first to (a) of FIG. 14, (a) of FIG. 14 shows an example in which part of a prediction interval 1400 is set as a caching interval 1420 based on the previously acquired user's biometric information (e.g., 60 kg). Here, if the range of a caching interval set by the reference value is 20 kg, then an interval from 40 kg, which is less than the reference value by 20 kg, to 80 kg, which is greater than the reference value by 20 kg, may be set as the caching interval for body weight.

In this case, the cloud server 10 may infer a directionality of the user's biological characteristics over time according to the user's biological characteristics. Here, a directionality of the biological characteristic may be inferred based on a statistical result related to the biological characteristics.

Meanwhile, as described above, in a case where the user's age is in a teenager range, the user's weight may be in an increasing trend. Accordingly, the cloud server 10 may determine that the user's biometric characteristics are in an increasing trend. Therefore, the cloud server 10 may set the caching interval to increase further. Therefore, as shown in (b) of FIG. 14, the minimum and maximum values of the caching interval 1420 that are set according to the previously acquired user's biometric information may be increased from the initially set values. That is, the entire set caching interval 1420 may be moved in a direction of increasing body weight.

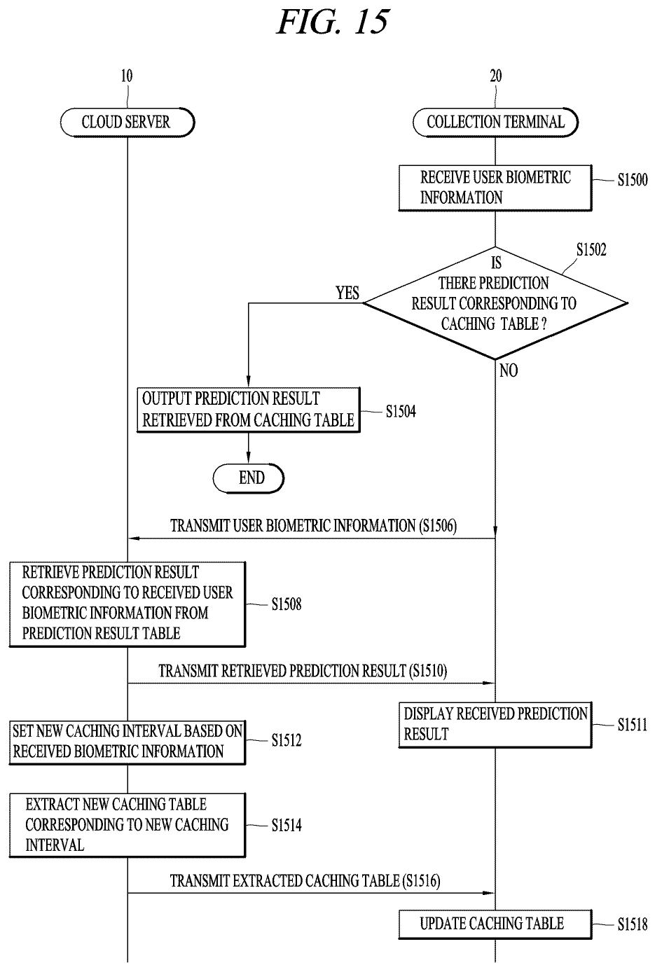

FIG. 15 is a flowchart showing an operation process of providing a diagnosis prediction result using a caching table in a health prediction system according to an embodiment of the present disclosure as described above.

Referring to FIG. 15, the collection terminal 20 of the health prediction system according to an embodiment of the present disclosure may first receive the user's biometric information from the measurement device (S1500). Furthermore, the collection terminal 20 may detect whether a caching table received from the cloud server 10 has a prediction rate corresponding to the user's biometric information received in the step S1500 (S1502).

For example, as described in FIG. 13, when the caching table stored in the collection terminal 20 is a prediction rate of the development of diabetes-related diseases predicted based on the user's weight and height, the collection terminal 20 may receive the user's height and weight from the measurement device in the step S1500. Furthermore, it may be distinguished whether the prediction rate corresponding to the received height and weight is included in the caching table. As an example, the collection terminal 20 may determine whether there is a prediction rate corresponding to the user's biometric information received in the step S1500 in a caching table depending on whether each caching interval constituting the caching table includes the biometric information received in the step S1500.

Furthermore, as a result of the discrimination in the step S1502, when a prediction rate corresponding to the biometric information acquired in the step S1500 can be detected through the caching table, the collection terminal 20 may output a prediction result according to the prediction rate retrieved from the caching table (S1504). In this case, the collection terminal 20 may apply a normally set threshold. Accordingly, when the detection of a prediction rate corresponding to the biometric information acquired through the caching table is allowed, there is no need to connect to the cloud server 10 through communication, and thus a disease prediction result corresponding to the acquired biometric information may be output very quickly. In addition, the computational load of the cloud server 10 may be reduced.

In this manner, when the prediction result is provided using the caching table, a change in threshold according to the user's context may be carried out by the collection terminal 20.

In this case, the collection terminal 20 may store information on a variation displacement of the threshold corresponding to a preset context. Furthermore, when the user's context calculated based on information collected from at least one peripheral device including the user himself or herself corresponds to the preset context, a threshold may be changed based on the variation displacement of the threshold corresponding to the preset context.

Furthermore, the collection terminal 20 may detect a prediction rate corresponding to the biometric information received from the measurement device based on the caching table. Furthermore, a prediction result determined based on the detected prediction rate and the changed threshold may be output through the output unit 250.

However, as a result of the discrimination in the step S1502, when a prediction rate corresponding to the biometric information acquired in the step S1500 cannot be detected through the caching table, the collection terminal 20 may transmit the biometric information acquired in the step S1500 to the cloud server 10 (S1506). Then, the cloud server 10 may retrieve a prediction rate corresponding to the biometric information transmitted from the collection terminal 20 from a prediction result table according to the universal prediction interval (S1508). Then, based on a preset threshold, it may be determined whether the retrieved prediction rate predicts a disease, and the determined result may be transmitted to the collection terminal 20 as a prediction result (S1510). Then, the collection terminal 20 may output the received prediction result through the output unit 250.

Meanwhile, when a prediction rate corresponding to the user's biometric information is not detected by the transmitted caching table, the cloud server 10 may re-set at least one caching interval based on the biometric information acquired in the step S1506 (S512). Furthermore, from the prediction result table, a new caching table including prediction rates corresponding to the reset caching interval may be extracted (S1516). Furthermore, the extracted new caching table may be transmitted to the collection terminal 20 (S1516). Then, the collection terminal 20 may update the caching table by replacing the previously stored caching table with the caching table received in the step S1516 (S1518).

Meanwhile, according to the foregoing description, it has been mentioned that the health prediction system according to an embodiment of the present disclosure is connected to a blockchain network so as to allow an authorized third party to view the user's biometric information stored in the cloud server 10 through the blockchain network.

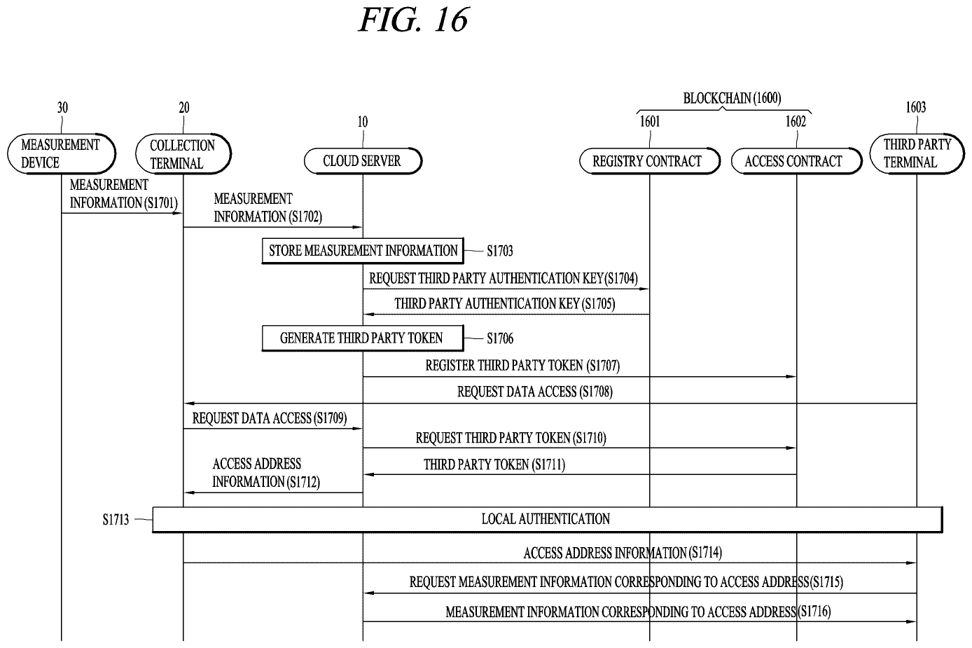

FIG. 16 is a flowchart showing an operation process for allowing an authorized third party to view a user's medical information in a health prediction system according to an embodiment of the present disclosure as described above.

Referring to FIG. 16, first, the measurement device 30 may transmit measured user's biometric information (measurement information) to a user terminal, that is, the collection terminal 20 (S1701). Then, the collection terminal 20 may transmit the collected measurement information to the cloud server 10 (S1702). Furthermore, the cloud server 10 may classify the received measurement information according to classification criteria such as a time at which the measurement information is received or a kind of biometric information, and store the classified measurement information in the biometric information Db 133 (S1703).

Meanwhile, when the user's biometric information is stored, the cloud server 10 may request at least one third party's authentication key that can access the user's biometric information from a blockchain network 1600. Here, the third party who can access the user's biometric information, which is a third party previously registered by the user, may be a medical service provider, trainer, or family member of the user.

In this case, the third party's authentication key request may be transmitted to a registry contract that constitutes the blockchain network 1600 (S1704). The registry contract 1601, which is an element of a blockchain to map data corresponding to an ID, may map a blockchain address corresponding to the ID. Therefore, the registry contract 1601 may provide, when requesting at least one third party's authentication key previously registered by the user from the cloud server 10, the address information of the requested at least one third party authentication key from data blocks distributively stored in a blockchain manner, and the cloud server 10 may receive at least one third party's authentication key based on the provided address information of the third party authentication key (S1705).

Then, the cloud server 10 may generate a token corresponding to at least one third party for which the authentication key is received (S1706). Here, the token represents an authority for the third party to view the user's biometric information, and only the third party with the token may be allowed to view the biometric information stored in the cloud server 10.

Here, the token may include a range of biometric information stored in the cloud server 10 that can be viewed by the third party (limitation on viewing scope), and may include a validity period (limitation on viewing period). Therefore, even if a token is registered, viewing may be allowed only within a scope specified in the token (e.g., a time at which the biometric information is stored or a kind of biometric information), and when the validity period has elapsed, it may not be allowed to view the user's biometric information through the token.

Meanwhile, when a token for the third party is generated in the step S1706, the cloud server 10 may register the generated token with the blockchain network 1600 (S1707). In this case, the token may be transmitted to an access contract 1602 that constitutes the blockchain network 1600. Here, the access contract 1602 is an element that manages an access authority to data blocks in the blockchain network 1600, and the token as an access authority to biometric information stored in the cloud server 10 may be registered with the access contract 1602.

Meanwhile, when the third party's token is registered with the access contract 1602 in the step S1707, the third party with a legitimate authority to view the user's biometric information, that is, the third party designated in advance by the user (hereinafter, referred to as an authorized third party) may transmit a request for access to user biometric information to the user terminal 20 through his or her own terminal 1603 (S1708). Then, the user terminal 20 may transmit the data access request received from the authorized third party to the cloud server 10 (S1709).

Then, the cloud server 10 may request the third party's token corresponding to the received data access request from the access contract 1602 in which the token is registered (S1710). Furthermore, in response to the token request, the access contract 1602 may transmit the third party's token corresponding to the data access request to the cloud server 10 (S1711). Then, the cloud server 10 may check the third party's viewing scope and viewing period through the token received from the access contract 1602, and transmit, when the user biometric information requested by the third party is within the viewing scope and viewing period allowed by the token, address information, that is, access address information, that can access an area where biometric information corresponding to the third party's data access request is stored, to the user terminal 20 (S1712).

Then, the user terminal 20 may perform additional authentication on the third party's terminal 1603 (S1703). For example, the user terminal 20 may determine whether the third party's terminal 1603 is located within a preset distance from the user terminal 20 or within the same area. As a result of the local authentication, only if the third party's terminal 1603 is located within a preset distance from the user terminal 20 or within the same area, the user terminal 20 may transmit the access address information received from the cloud server 10 to the third party terminal 1603 (S1714).

Then, the third party terminal 1603 may request to view some of the user's biometric information stored in the cloud server 10, which is allowed to be viewed by the token, based on the received access address information (S1715). Then, the cloud server 10 may allow viewing according to the viewing request, and as a result, some of the allowed user's biometric information stored in the cloud server 10 may be displayed through the third party terminal 1603 (S1716).

Meanwhile, even if the user's biometric information can be provided to the third party terminal 1603 through viewing with respect to the access address transmitted in the step S1716, it may be limited to viewing. That is, the third party terminal 1603 may only be allowed to check the user's biometric information, but the processing or storing of the user's biometric information in the third party terminal 1603 may be limited. This is because the user's biometric information provided from the cloud server 10 to the third party terminal 1603 is only allowed to be viewed, and the biometric information is not actually provided.

The foregoing present disclosure may be implemented as computer-readable codes on a program-recorded medium. The computer-readable medium may include all types of recording devices each storing data readable by a computer system. Examples of the computer-readable media can include a hard disk drive (HDD), a solid state disk (SSD), a silicon disk drive (SDD), a ROM, a RAM, a CD-ROM, a magnetic tape, a floppy disk, and an optical data storage device, and the like, and also include a device implemented in the form of a carrier wave (for example, transmission via the Internet). In addition, the computer may be the server 10 of the health prediction system according to an embodiment of the present disclosure.

The above detailed description is therefore to be construed in all aspects as illustrative and not restrictive. The scope of the present disclosure should be determined by reasonable interpretation of the appended claims and all changes that come within the equivalent scope of the present disclosure are included in the scope of the present disclosure.

1. A server of a health prediction system, the server comprising:
a communication unit that performs wireless communication with a collection terminal that collects a user's biometric information measured by at least one measurement device;
an artificial intelligence unit that generates, when the characteristic information of the at least one measurement device is received through the communication unit, a prediction model for a specific disease by reflecting the received characteristic information of the measurement device, and performs training on the prediction model based on the collected user's biometric information; and
a controller that controls, when training on the prediction model is carried out, the artificial intelligence unit to calculate a prediction rate related to the specific disease in response to biometric information collected in the collection terminal from the trained prediction model, discriminates a prediction result for the specific disease based on the calculated prediction rate and a preset threshold, and transmits the prediction result to the collection terminal such that the discriminated prediction result is displayed through the collection terminal.

2. The server of claim 1, wherein the collection terminal acquires information related to a user's context from at least one peripheral device around the user, analyzes the user's context based on the acquired information, and transmits context information on the analyzed user's context to the server, and
wherein the server changes a threshold based on the context information, and discriminates a prediction result for the specific disease based on the changed threshold and the prediction rate.

3. The server of claim 2, wherein the at least one peripheral device is information on at least one mobile terminal located around the user or an access point (AP) provided in a region where the user is located.

4. The server of claim 1, wherein the prediction model is an artificial neural network comprising at least one exclusive layer, which is a hidden layer comprising weights applied to input biometric information classified into a specific group, and at least one common layer, which is a hidden layer comprising weights applied to all input biometric information, and
wherein the biometric information is input to either one of the exclusive layer or the common layer depending on whether it is classified into the specific group, and a prediction rate output as a result of the prediction model is output from any one of the at least one common layer.

5. The server of claim 4, wherein the controller inputs the biometric information as the biometric information of the specific group into the prediction model according to the characteristic information of a measurement device that measures the biometric information.

6. The server of claim 1, wherein the characteristic information of the measurement device comprises at lase one of a manufacturer of the measurement device, a kind of biometric information measured by the measurement device, a type of the measurement device, a mounting method in which the measurement device is mounted by a user, and a measurement method in which the user's biometric information is measured by the measurement device.

7. The server of claim 6, wherein the characteristic information of the measurement device comprises quantified clinical accuracy that quantifies the clinical accuracy of the measurement device.

8. The server of claim 1, wherein the controller updates the prediction model when a preset update condition is satisfied, and
wherein the preset update condition is satisfied when a preset update cycle is expired or when a measurement device that measures the user's biometric information is added, replaced, or any one measurement device is removed.

9. The server of claim 8, wherein the controller updates the prediction model to change a weight assigned to at least one node among hidden layers constituting the prediction model, or to remove at least one of the hidden layers or further include a new hidden layer including nodes to which a new weight is assigned, and determines the changed weight based on the device characteristic information of the added, replaced or removed measurement device, or determines the hidden layer to be removed or the new hidden layer.

10. The server of claim 8, wherein the controller generates at least one new prediction model according to the device characteristic information of the added, replaced, or removed measurement device, and calculates a statistical value of prediction rates calculated from the existing prediction model and the at least one new prediction model, respectively, with respect to the collected user's biometric information as the prediction rate.

11. The server of claim 8, wherein the controller updates the trained prediction model by combining at least one prediction model derived from a training process for the prediction model based on the collected user's biometric information with the trained prediction model.

12. The server of claim 11, wherein the controller compares the weights of hidden layers corresponding to one another from the trained prediction model and the at least one derived model, respectively, and adds nodes with different weights to combine the trained prediction model with the at least one derived model.

13. The server of claim 1, wherein when the measurement device is a measurement device that measures a user's blood glucose level, the prediction model is a model that has trained a variation in the user's blood glucose level that varies depending on each nutrient, and
wherein when a user inputs a virtual diet through a collection terminal, the controller analyzes the nutrients of each food included in the input diet, calculates a variation in the user's total blood glucose level corresponding to the virtual diet based on a variation in the user's blood glucose level according to the analyzed nutrients, respectively, and an intake amount of each food that is input with the virtual diet, and transmits the calculated variation to the collection terminal in response to an input of the virtual diet.

14. The server of claim 2, wherein when the training of the prediction model is completed, the controller determines a range of the minimum and maximum values of the user's biometric information that can be detected through the measurement device from the user's basic biometric characteristics, controls the artificial intelligence unit to calculate the prediction rates of the prediction model corresponding to each biometric information included in the determined range of the minimum and maximum values of the biometric information, and transmits some of the calculated prediction rates to the collection terminal as a caching table.

15. The server of claim 14, wherein when the caching table is received from the server, the collection terminal detects a prediction rate corresponding to a bio-signal collected from the measurement device from the caching table, discriminates a prediction result for the specific disease based on the prediction rate detected from the detected caching table and a threshold according to the context information, and outputs the discriminated prediction result.

16. The server of claim 1, wherein the server further comprises a memory in which the user's biometric information collected through the communication unit is classified and stored according to a time at which the biometric information is measured and a kind of the biometric information, and
wherein the controller provides address information related to a storage area where the classified biometric information is stored in the memory in response to a user's request.

17. The server of claim 16, wherein the controller registers a token representing the authority of a third party to request the user's biometric information through a blockchain network, provides the address information to the third party at the user's request based on the registered token, and limits a scope of the user's biometric information that can be viewed by the third party and a validity period during which the user's biometric information can be viewed based on the token registered in the blockchain network.

18. A method of controlling a health prediction system that provides a prediction result related to a specific disease based on a user's biometric information, the method comprising:
connecting, by a collection terminal of the health prediction system, to at least one measurement device through communication to measure the user's biometric information, and collecting the device characteristic information of the at least one measurement device;
receiving, by a cloud server, the characteristic information of the measurement device collected from the collection terminal, and generating a prediction model for a specific disease on which the characteristic information of the measurement device is reflected;
receiving, by the collection terminal, the user's biometric information collected by the at least one measurement device;
receiving, by the cloud server, biometric information collected by the collection terminal, and training the prediction model based on the received biometric information;
calculating, by the cloud server, when the training of the prediction model is completed, a prediction rate corresponding to the biometric information received from the collection terminal from the trained prediction model; and
discriminating, by the cloud server or the collection terminal, a prediction result for the specific disease based on a preset threshold and the prediction rate, and outputting the discriminated prediction result.

19. The method of claim 18, wherein the characteristic information of the measurement device comprises at least one of a manufacturer of the measurement device, a kind of biometric information measured by the measurement device, a type of the measurement device, a mounting method in which the measurement device is mounted by a user, and a measurement method in which the user's biometric information is measured by the measurement device.

20. The method of claim 18, wherein the calculating of a prediction rate comprises:
determining, by the cloud server, a range of the minimum and maximum values of the user's biometric information that can be detected through the measurement device from the user's basic biometric characteristics;
calculating, by the cloud server, the prediction rates of the trained prediction model corresponding to each biometric information included in the range of the minimum and maximum values of the biometric information; and
transmitting, by the cloud server, some of the calculated prediction rates to the collection terminal as a caching table, and
wherein the outputting, by the cloud server or the collection terminal, of the prediction result comprises:
detecting, by the collection terminal, any one prediction rate corresponding to biometric information received from the measurement device among prediction rates included in the caching table; and
discriminating, by the collection terminal, a prediction result for the specific disease based on the detected prediction rate and a preset threshold, and outputting the discriminated prediction result.
